SEQUENCE LISTING (2 sequences)

SEQ ID NO: 1 - DNA, 58 bp, Artificial sequence
  Synthetic construct
ccggcttcaacgtctctcaacagatctcgagatctgttgagagacgttgaagtttttg

SEQ ID NO: 2 - DNA, 59 bp, Artificial sequence
  Synthetic construct
ccggtcaagacgctggaaagtattactcgagtaatactttccagcgtcttgattttttg

(19) United States
(12) Patent Application Publication
(10) Pub. No.: US 2023/0039520 A1
(43) Pub. Date: Feb. 9, 2023

(54) PERICYTE-SPARING THERAPY

(71) Applicant: The Board of Trustees of the Leland Stanford Junior University, Stanford, CA (US)

(72) Inventors: Ansuman SATPATHY, Stanford, CA (US); Howard Y. CHANG, Stanford, CA (US); Kevin R. PARKER, Stanford, CA (US)

(21) Appl. No.: 17/793,024

(22) PCT Filed: Jan. 14, 2021
(86) PCT No.: PCT/US2021/013492

§ 371 Date: Jul. 14, 2022

Related U.S. Application Data
(60) Provisional application No. 62/961,620, filed on Jan. 15, 2020.
(60) Provisional application No. 63/080,582, filed on Sep. 18, 2020.

(51) Int. Cl. C07K 16/28; C12N 15/113; C07K 14/705; A61P 25/28; C07K 16/44

(52) U.S. Cl. CPC C07K 16/2818; C07K 16/2803; C12N 15/1138; C07K 14/70532; A61P 25/28; C07K 16/289; C07K 16/2896; C07K 16/44; C07K 2317/622; C07K 2317/569; C12N 2310/14; C12N 2310/531; C07K 2317/31

(57) ABSTRACT

Methods and systems to reduce neurotoxicity associated with the treatment of CD19⁺ B-cell hyperproliferative disorders are disclosed. Neurotoxicity is reduced by the use of agents that protect CD19⁺ neurovascular pericytes and/or CD19⁺ vSMCs from attack by CD19-targeted therapy, and by modification of CD19-targeted therapy to avoid CD19⁺ pericytes and/or CD19⁺ vSMCs.

### CROSS-REFERENCE TO RELATED APPLICATIONS

This application claims priority to U.S. Provisional Patent Application Nos. 62/961,620, filed Jan. 15, 2020, and 63/080,582, filed Sep. 18, 2020, the disclosures of which are incorporated by reference herein in their entireties, including any drawings.

### FEDERALLY SPONSORED RESEARCH AND DEVELOPMENT

This invention was made with government support under grant nos. P50-HG007735 and K08-CA23188-01 awarded by The National Institutes of Health. The government has certain rights in the invention.

### INCORPORATION OF THE SEQUENCE LISTING

This application contains a Sequence Listing, which is hereby incorporated herein by reference in its entirety. The accompanying Sequence Listing text file, named “078430_511001WO_Sequence_Listing_ST25” was created on Jan. 13, 2021 and is 740 B.

### BACKGROUND OF THE INVENTION

CD19 is a surface antigen that is expressed on all B-lineage cells (except for plasma cells and follicular dendritic cells), and is typically highly expressed in B-lineage cancers such as acute lymphoblastic leukemia (ALL) and chronic lymphocytic leukemia (CLL). CD19 expression is so characteristic of B-lineage cells that it is often used as a biomarker for B-cells. Accordingly, it makes an attractive target for treating B-cell derived lymphomas and leukemias.

For patients with B-cell lymphoma, including those who have relapsed after receiving traditional chemotherapy regimens, immunotherapeutic approaches have shown tremendous clinical efficacy. In a recent Phase II study of 111 patients with refractory B-cell lymphoma, of whom 101 were administered CAR-T cell therapy targeting CD19, 40% of patients showed complete remission of disease 15 months after treatment (S. S. Neelapu et al., N Engl J Med (2017) 377:2531-44). Similar results were observed in a separate study, with complete remission observed in 43% and 71% of patients with diffuse large B-cell and follicular lymphoma, respectively (S.J. Schuster et al., N Engl J Med (2017) 377:2545-54). However, specific anti-CD19 therapy can have substantial adverse side effects: in addition to effects related to cytokine release syndrome (CRS), both studies reported a high incidence of neurotoxicity (64% and 39%, respectively), in agreement with previously reported rates of neurotoxicity in patients receiving CD19 CAR-T cell therapy and CD19/CD3 BiTE® (bi-specific T cell engager) therapy (J. Gust et al., Cancer Discov (2017) 7:1404-19; K. A. Hay et al., Blood (2017) 130:2295-306; J. Gust et al., CNS Drugs (2018) 32:1091-101; M. E. Goebeler et al., J Clin Oncol (2016) 34(10):1104-11; A. Viardot et al., Blood (2016) 127:1410-16). The mechanism for such neurotoxicity has not been reported.

Methods and agents for reducing the neurotoxicity associated with highly effective anti-CD19 therapies such as CAR-T and BiTE® are urgently needed, in order to fully and safely employ these therapies. Alternatively, there is an urgent need to identify the source of this neurotoxicity, and to design highly effective anti-CD19 therapies that reduce or eliminate treatment-associated neurotoxicity.

All publications and patent applications mentioned in this disclosure are herein incorporated by reference to the same extent as if each individual publication or patent application was specifically and individually indicated to be incorporated by reference. No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

### BRIEF SUMMARY OF THE INVENTION

As set forth herein, CD19 is present not only on the surface of B-cells, but has now been found to be expressed in a subset of pericytes and vascular smooth muscle cells (vSMCs) found in cerebral vasculature. Anti-CD19 therapies can damage neurovascular pericytes and vascular smooth muscle cells (vSMCs) and disrupt or damage the blood-brain barrier (BBB), which permits cytotoxic T cells and other agents to enter the brain with deleterious and toxic effects. Provided herein are protective methods and reagents that reduce toxicity to pericytes and vascular smooth muscle cells (vSMCs) and damage to the BBB.

An aspect of the disclosure is a method for aiding in the treatment of a B-cell hyperproliferative disorder in a subject using a CD19-targeted therapy, by (a) administering an effective amount of an agent to the subject, wherein the agent is selected from a binding agent which binds to CD19 and reduces binding of the CD19-targeted therapy to CD19⁺ neurovascular pericytes and/or CD19+ vSMCs; or an expression modulator that down-regulates or eliminates the expression of CD19 by neurovascular pericytes and/or vSMCs; or a bispecific binding agent that comprises a first binding domain that activates an immune checkpoint surface protein, and a second binding domain having affinity for a non-CD19 pericyte and/or vSMC surface protein; and (b) administering a therapeutically effective amount of the CD19-targeted therapy. In some embodiments, the agent is a binding agent that comprises an antibody or an antibody derivative that binds to CD19. In some embodiments, the binding agent is an antibody derivative. In some embodiments, the antibody derivative is a nanobody, duobody, diabody, triabody, minibody, F(ab′)₂ fragment, Fab fragment, single chain variable fragment (scFv), or a single domain antibody (sdAb). In some embodiments, the binding agent is a nanobody or an scFv.

In some embodiments, the agent is an expression modulator that down-regulates or eliminates expression of CD19 by neurovascular pericytes and/or vSMCs and comprises an antisense oligonucleotide (ASO), an siRNA, or an shRNA. In an embodiment, the expression modulator agent is a single stranded ASO. In an embodiment, the expression modulator agent is an siRNA. In an embodiment, the expression modulator agent is an shRNA.

In some embodiments, the agent is administered intrathecally.

In some embodiments, the agent is a bispecific binding agent, and the immune checkpoint surface protein comprises CTLA-4, A2AR, VTCN1, BTLA, PD-1, or TIM-3. In an embodiment, the first binding domain comprises a CTLA-4-binding domain of CD80 or CD86. In an embodiment, the first binding domain comprises a CTLA-4 agonist. In some embodiments, the CTLA-4 agonist is an antibody or an antibody derivative. In some embodiments, the first binding domain comprises a PD-1 binding domain of PD-Ll or PD-L2, or an antibody or an antibody derivative specific for PD-1. In an embodiment, the first binding domain is a PD-1 agonist.

In some embodiments, the first binding domain comprises an A2AR binding agent. In some embodiments, the A2AR binding agent is an antibody or antibody derivative specific for A2AR, or an A2AR agonist.

In some embodiments, the non-CD19 pericyte and/or vSMC surface protein comprises BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3. In some embodiments, the second domain of the bispecific binding agent is an antibody or a derivative thereof specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3. In some embodiments, a canonical vSMC marker is ACTA2.

In some embodiments, the agent is administered at about the time of administering the CD19-targeted therapy. In other embodiments, the agent is administered about 15, 14, 13, 12, 11, 10, 9, 8, 7, 6, 5, 4, 3, 2 or 1 days prior to administering the CD19-targeted therapy. In other embodiments, the agent is administered about 1, 2, 3, 4, 5, 6, 7, 8, 9, or 10 days after administering the CD19-targeted therapy. In some embodiments, the agent is administered multiple times between about 15 days prior and about 10 days after administering the CD19-targeted therapy. In some embodiments, the agent is administered after the subject presents signs of neurotoxicity.

In some embodiments, the agent reduces the number of neurovascular pericytes and/or vSMCs that are killed or incapacitated by the CD19-targeted therapy in vitro by about 10%, about 20%, about 30%, about 40%, about 50%, about 60%, about 70%, about 80%, about 90%, or about 100%. In some embodiments, the agent reduces the number of neurovascular pericytes and/or vSMCs that are killed or incapacitated by the CD19-targeted therapy in an in vivo animal model by about 10%, about 20%, about 30%, about 40%, about 50%, about 60%, about 70%, about 80%, about 90%, or about 100%. In some embodiments, the agent reduces the disruption of the blood-brain barrier (BBB) by the CD19-targeted therapy by about 10%, about 20%, about 30%, about 40%, about 50%, about 60%, about 70%, about 80%, about 90%, or about 100%, as measured in an in vivo animal model using exclusion of a marker as a measure of BBB permeability. In an embodiment, the marker is Evans Blue dye.

In some embodiments, the agent is administered as a formulation that comprises an agent that binds to CD19 and reduces binding of the CD19-targeted therapy to CD19⁺ neurovascular pericytes and/or CD19⁺ vSMCs, or down-regulates the expression of CD19 by pericytes and/or vSMCs; and a carrier suitable for intrathecal administration.

In some embodiments, the CD19-targeted therapy comprises an anti-CD19 chimeric antigen receptor T cell (CAR-T) or a bispecific T cell engager specific for CD19.

Another aspect is a formulation that comprises an agent that binds to CD19 and reduces binding of the CD19-targeted therapy to CD19⁺ neurovascular pericytes and/or CD19⁺ vSMCs, or down-regulates the expression of CD19 by pericytes and/or vSMCs; and a carrier suitable for intrathecal administration. In some embodiments, the formulation does not contain an antimicrobial agent or a preservative.

Another aspect is a system for treating a B-cell hyperproliferative disorder in a subject, while preserving the subject's BBB, the system comprising a CD19-targeted therapy, and an agent selected from a binding agent which binds to CD19 and reduces binding of the CD19-targeted therapy to CD19⁺ neurovascular pericytes and/or CD19⁺ vSMCs; an expression modulator that down-regulates or eliminates the expression of CD19 by neurovascular pericytes and/or vSMCs; and a bispecific binding agent that comprises a first binding domain that activates an immune checkpoint surface protein, and a second binding domain having affinity for a non-CD19 and/or vSMC pericyte surface protein. In some embodiments, the CD19-targeted therapy comprises an anti-CD19 chimeric antigen receptor T cell (CAR-T) or a bispecific T cell engager specific for CD3 and CD19. In some embodiments, the agent comprises an antibody or an antibody derivative that binds to CD19. In some embodiments, the antibody derivative is a nanobody, duobody, diabody, triabody, minibody, F(ab′)₂ fragment, Fab fragment, single chain variable fragment (scFv), or a single domain antibody (sdAb). In some embodiments, the agent comprises a nanobody or an scFv.

In some embodiments, the agent is an expression modulator and comprises an antisense oligonucleotide (ASO), an siRNA, or a shRNA. In an embodiment, the agent comprises an siRNA. In an embodiment, the agent comprises an shRNA. In an embodiment, the agent comprises an siRNA. In an embodiment, the agent comprises an ASO. In some embodiments of the system, the agent is provided in a formulation suitable for intrathecal administration.

In some embodiments of the system, the agent is a bispecific binding agent and the immune checkpoint surface protein comprises CTLA-4, A2AR, VTCN1, BTLA, PD-1, or TIM-3. In some embodiments, the first binding domain comprises a CTLA-4 agonist. In some embodiments, the CTLA-4 agonist is an antibody or an antibody derivative. In some embodiments, the first binding domain comprises a PD-1 binding domain of PD-L1 or PD-L2, or an antibody or an antibody derivative specific for PD-1. In an embodiment, the first binding domain is a PD-1 agonist. In some embodiments, the first binding domain comprises an A2AR binding agent. In some embodiments, the A2AR binding agent is an antibody or antibody derivative specific for A2AR, or an A2AR agonist.

In some embodiments of the system, the non-CD19 pericyte and/or vSMC surface protein comprises BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3. In some embodiments, the second domain of the bispecific binding agent is an antibody or a derivative thereof specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3. In some embodiments, a canonical vSMC marker is ACTA2.

In some embodiments of the system, the agent is encoded in a vector. In some embodiments, the vector is an expression vector having a promoter that is functional in a mammalian cell, and is operably linked to a nucleic acid that encodes the agent. In some embodiments of the system, the agent is provided in a pharmaceutically acceptable formulation. In some embodiments, the formulation is acceptable for intrathecal or intracerebral administration. In some embodiments, the agent is not cytotoxic to neurovascular pericytes and/or vSMCs.

Another aspect of the disclosure is a bispecific binding agent for reducing the potential neurotoxicity of a CD19-targeted therapy, wherein the bispecific binding agent comprises a first binding domain that activates an immune checkpoint surface protein, and a second binding domain that specifically binds a non-CD19 neurovascular pericyte and/or vSMC surface protein. In some embodiments, the immune checkpoint surface protein comprises CTLA-4, A2AR, VTCN1, BTLA, PD-1, or TIM-3. In some embodiments, the first binding domain comprises a CTLA-4-binding domain of CD80 or CD86, or comprises a CTLA-4 agonist. In some embodiments, the CTLA-4 agonist comprises an antibody or an antibody derivative. In some embodiments, the first binding domain comprises a PD-1 binding domain of PD-L1 or PD-L2, or an antibody or a derivative thereof specific for PD-1. In an embodiment, the first binding domain comprises a PD-1 agonist. In some embodiments, the first binding domain comprises an A2AR binding agent. In some embodiments, the A2AR binding agent is an antibody or antibody derivative specific for A2AR, or an A2AR agonist. In some embodiments of the bispecific binding agent, the non-CD19 pericyte and/or vSMC surface protein comprises BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

Another aspect is a nucleic acid that encodes a bispecific binding agent of the disclosure. In some embodiments, the nucleic acid is contained within a vector. In some embodiments, the vector is an expression vector having a promoter that is functional in a mammalian cell, and is operably linked to a nucleic acid that encodes the bispecific binding agent.

Another aspect is a formulation for use in connection with CD19-targeted therapy, the formulation comprising an effective amount of the bispecific binding agent or a nucleic acid encoding the bispecific binding agent, and a pharmaceutically acceptable carrier. In some embodiments, the effective amount is sufficient to reduce the number of neurovascular pericytes and/or vSMCs that are killed or incapacitated by the CD19-targeted therapy in vitro by about 10%, about 20%, about 30%, about 40%, about 50%, about 60%, about 70%, about 80%, about 90%, or about 100%. In some embodiments, the agent reduces the number of neurovascular pericytes and/or vSMCs that are killed or incapacitated by the CD19-targeted therapy in an in vivo animal model by about 10%, about 20%, about 30%, about 40%, about 50%, about 60%, about 70%, about 80%, about 90%, or about 100%. In some embodiments, the agent reduces the disruption of the blood-brain barrier (BBB) by the CD19-targeted therapy by about 10%, about 20%, about 30%, about 40%, about 50%, about 60%, about 70%, about 80%, about 90%, or about 100%, as measured in an in vivo animal model using exclusion of a marker as a measure of BBB permeability. In an embodiment, the marker is Evans Blue dye.

Another aspect is a method for aiding in the treatment of a B-cell hyperproliferative disorder in a subject, by administering an effective amount of a formulation of the disclosure, a system of the disclosure, or a bispecific binding agent of the disclosure.

Another aspect is a kit for the treatment of a B-cell hyperproliferative disorder in a subject, comprising a formulation of the disclosure, a system of the disclosure, a bispecific binding agent of the disclosure, or a nucleic acid encoding a bispecific binding agent of the disclosure; together with instructions for the use thereof. An embodiment is the kit wherein the instructions are printed. In some embodiments, the nucleic acid is contained within a vector.

Another aspect is the use of a formulation of the disclosure, a system of the disclosure, a bispecific binding agent of the disclosure, a nucleic acid encoding a bispecific binding agent of the disclosure, or a kit of the disclosure. In some embodiments, the use is the treatment of a B-cell hyperproliferative disorder in a subject.

Another aspect is the use of a formulation of the disclosure, a system of the disclosure, a bispecific binding agent of the disclosure, a nucleic acid encoding a bispecific binding agent of the disclosure, or a kit of the disclosure, for the manufacture of a medicament. In some embodiments, the medicament is for the treatment of a B-cell hyperproliferative disorder in a subject.

### BRIEF DESCRIPTION OF THE DRAWINGS

FIG. 1 shows non-neuronal, non-erythroid cells from 2,364 human prefrontal cortex cells clustered into broad populations using UMAP based on single-cell prefrontal cortex RNA-sequencing data. These were segregated into astrocyte, lymphocyte, microglial, oligodendrocyte precursor (OPC), endothelial, vascular smooth muscle cells (vSMCs) and pericyte/endothelial populations.

FIG. 2A shows the cells expressing CD19 based on the same data shown in FIG. 1. FIG. 2B shows that the same cells expressing CD19 expressed CD248, indicating they are CD248 positive pericytes. FIG. 2C shows that the same cells expressing CD19 and CD248 lacked ACTA2 expression, indicating that these cells are pericytes.

FIG. 3 shows that a subset of the cells that expressed CD248 and RGS5 (i.e., markers for pericytes and vSMCs) also expressed CD19, and that there was no detectable expression of CD79A (a B-cell marker) in any of the identified pericytes.

FIG. 4 shows that a subset of the cells from human ventral midbrain that expressed CD248 also expressed CD19, with no detectable expression of CD79A, and that cells that expressed PECAM1 (an endothelial marker) had no detectable expression of CD79A. Only a few endothelial cells exhibited CD19 expression (believed to be the result of pericyte-endothelial cell doublets, as pericytes and endothelial cells strongly adhere to each other).

FIG. 5 shows that mice treated with anti-murine CD19 CAR-T cells comprising a CAR having an scFv specific for murine CD19 with a 4-1BB/CD3ζ endodomain (moCD19BBz) or a CD28/CD3ζ endodomain (moCD1928z) displayed BBB extravasation indicative of disruption of the BBB, while a corresponding anti-human CD19 CAR-T (hCD19BBz) had little or no effect.

FIG. 6 shows that mice treated with murine T cells expressing murine versions of the CARs in FIG. 5 also displayed BBB extravasation indicative of disruption of the BBB.

FIG. 7 is a histogram of mean abundance of all genes in the human prefrontal cortex. Percentiles indicate the expression rank of genes relative to all detected genes.

FIG. 8 is a histogram of mean gene expression values in identified pericyte cells in human forebrain (10× Chromium V2). Relative gene expression percentiles are shown for indicated genes.

FIG. 9 is a histogram of mean gene expression values in identified pericyte cells in human forebrain (Fluidigm C1). Relative gene expression percentiles are shown for indicated genes.

FIGS. 10A-10F show that CD19 is expressed in both pericytes and vSMCs. FIG. 10A shows a subset of non-neuronal cells from Zhong 2018, La Manno 2016, La Manno 2018 datasets. FIG. 10B shows expression of marker genes used for clustering the subset of non-neuronal cells from the three dataset. Y-axis labels indicate maximum TPM value shown. FIG. 10C shows low expression of vSMC marker genes in the non-neuronal cell subset. Y-axis labels indicate maximum TPM value shown. FIG. 10D shows high expression of pericyte marker genes in the non-neuronal cell subset. Y-axis labels indicate maximum TPM value shown. FIG. 10E shows neurovascular and progenitor subset of the BICCN data annotated by brain region. FIG. 10F shows expression of vSMC marker (ACTA2) as well as CSPG4 (pericyte) and CLDN5 (endothelial) markers. CD19 is expressed primarily in the vSMC and pericyte clusters.

FIGS. 11A-11F show that the CAR-T recognized CD19 isoform is expressed in the adult human. FIG. 11A shows expression of CD248 and CD19 by different brain region in Allen Institute Brainspan dataset. FIG. 11B shows expression of CD248 and CD19 in the postnatal and adult human brain samples in Allen Institute Brainspan dataset. FIG. 11C shows a histogram of the distribution of correlation values for all genes with CD19 expression in postnatal sample. The indicated percentiles indicate the percentile of that gene's correlation. FIG. 11D shows enriched GO terms in the top 200 genes by spearman correlation with CD19. FIG. 11E shows gene score distribution in single cells belonging to pericyte or endothelial clusters, as well as other brain cells; along with B cells and other PBMCs. Gene score was calculated with the top 30 genes by spearman correlation. FIG. 11F shows RPKM values per exon of CD19 in the Brainspan data, showing expression of the key exons 2 and 4 for CAR-T cell recognition.

FIG. 12 shows a track plot showing expression of selected marker genes for each population (brain pericytes, lung pericytes, lung vSMCs, brain endothelial cells, and lung endothelial cells), showing brain pericyte-specific expression of CD19. CD19 expression is limited to brain pericytes, but not lung pericytes. Y-axis labels indicate maximum TPM value.

### DETAILED DESCRIPTION OF THE INVENTION

The present disclosure relates generally to methods for reducing damage to the blood-brain barrier (BBB) during CD19-targeted therapy, by reducing damage to CD19⁺ pericytes and/or CD19⁺ vSMCs located along neurovascular blood vessels that may otherwise occur during CD19-targeted therapy. The disclosure also relates to agents for reducing or avoiding damage to CD19⁺ pericytes and/or CD19⁺ vSMCs, and CD19-targeted therapy that is designed to reduce damage to CD19⁺ pericytes and/or CD19⁺ vSMCs, for example, a CD19-targeted CAR-T therapy or a CD19-targeted BiTE® therapy.

A. Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

As used herein, a “therapeutically effective amount” of an agent is an amount sufficient to provide a therapeutic benefit in the treatment or management of a disease or disorder, or to delay or minimize one or more symptoms associated with the disease or disorder. A therapeutically effective amount of an agent means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the disease or disorder, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

B-cell hyperproliferative disorders include B-cell leukemias and lymphomas such as acute lymphoblastic leukemia (ALL), chronic lymphocytic leukemia (CLL), B-cell prolymphocytic leukemia, precursor B lymphoblastic leukemia, hairy cell leukemia, diffuse large B-cell lymphoma, follicular lymphoma, marginal zone lymphoma, mantle cell lymphoma, Burkitt's lymphoma, MALT lymphoma, Waldenstrom's macroglobulinemia, and other disorders characterized by the overgrowth of B-lineage cells.

Mural cells are an integral part of the neurovascular unit (NVU), which surround endothelial cells and are critical for regulating the integrity of the BBB. Mural cells include pericytes and vSMCs, which are closely related cell types that differ anatomically: pericytes localize along capillaries, while vSMCs are found along larger vessels, including arteries, arterioles, and venules. These cells are transcriptionallyl similar, sharing the identity of any marker genes and appearing to exist on a transcriptional lineage continuum. ACTA2, encoding alpha-smooth muscle actin, is a canonical marker used to distinguish these two populations, which is significantly upregulated in vSMCs. Many pericyte markers, however, such as CSPG4 and RGS5, are also highly expressed in vSMCs, causing brain vSMCs to often be annotated as pericytes. Neurovascular pericytes are mesenchymal cells present along neurovascular capillary walls and post-capillary venules of the brain and spinal cord that are important for maintenance of the blood-brain barrier, regulation of immune cell entry to the central nervous system, control of blood flow in the brain, and are critical to maintaining the blood-brain barrier (BBB) (D. Attwell et al., J Cereb Blood Flow Metab (2016) 36:451-5; A. Armulik et al., Nature (2018) 468:557-61; E. A. Winkler et al., Nat Neurosci (2014) 14(11):1398-405). Loss of capillary pericyte coverage leads to compromised BBB function, resulting in leakage from the vasculature into the CNS. BBB compromise can permit, for example, infiltration of inflammatory cells and macromolecules that would otherwise be excluded. This has been proposed as a mechanism by which pericyte dysfunction promotes neuroinflammation. See, e.g., R. D. Bell et al., Neuron (2010) 68(3):409-27, reporting that loss of 20% of neurovascular pericytes in mice disrupted the BBB and caused vascular damage.

Pericytes can be identified by positive immuno-staining with antibodies specific for alpha smooth muscle actin (e.g., anti-alpha-sm1, Biomakor, Rehovot, Israel), HMW-MAA, and pericyte ganglioside antigens such as MAb 3G5 (Schlingemann et al., Am J Pathol (1990) 136: 1393-405); and, negative immuno-staining with antibodies to cytokeratins (epithelial and fibroblast markers) and von Willebrand factor (an endothelial marker). Both vascular smooth muscle cells (vSMCs) and pericytes are positive by immunostaining with the NR-AN-01 monoclonal antibody. Other characteristic markers include PDGFRβ, desmin, αSMA, and NG2 (US Patent Publication No. 2018/0044672A1). Vascular smooth muscle cells (vSMCs) can be identified by positive immuno-staining with antibodies specific for ACTA2 (smooth muscle actin-α). Pericytes have not previously been reported to express CD19. Since pericytes and vSMCs are not known to derive from B-cell lineages, expression of a B-cell lineage-specific marker like CD19 by pericytes and vSMCs is unexpected.

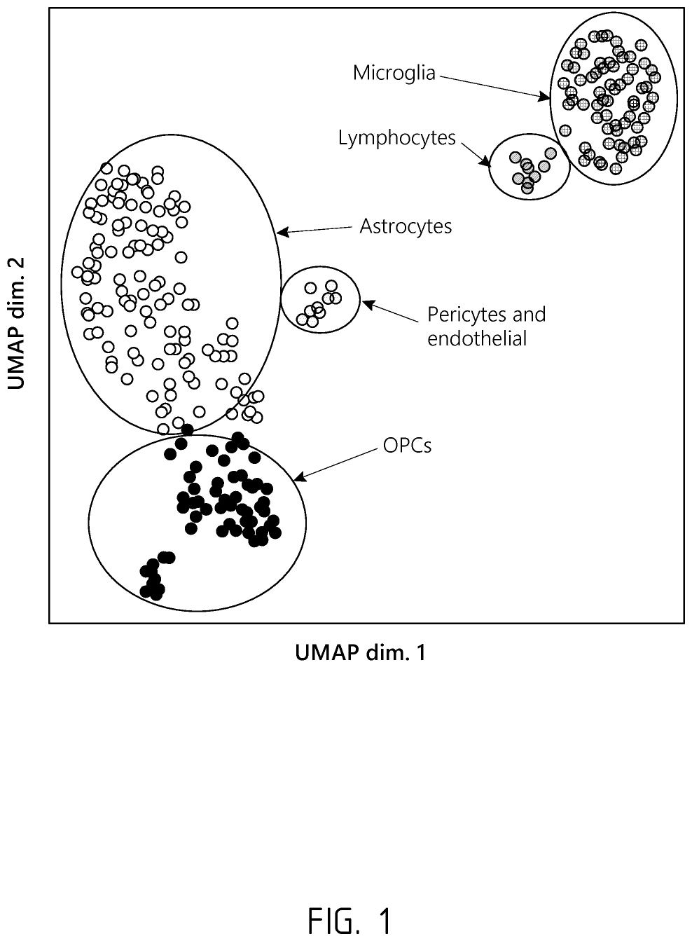

As set forth herein, the presence of CD19⁺ neurovascular pericytes and CD19⁺ vSMCs was determined by sorting single cell sequence data derived from brain tissue. As shown in FIGS. 1-4, this data demonstrates that pericytes and vSMCs are present in this tissue, that CD19 expression is associated with the pericytes and vSMCs, and that no other tissue or non-B-cell cell type is associated with CD19 expression in this data sample. CAR-T cells were then constructed that were specific for (a) murine CD19 (mCD19) or (b) human CD19 (hCD19). The sequences of mCD19 and hCD19 differ enough that CAR-T cells specific for one do not cross-react with the other. Lymphodepleted mice treated with Evans Blue dye followed by administration of mCD19 CAR-T cells exhibited staining (as observed by fluorescence microscopy) in the brain indicative of BBB disruption, whereas mice administered hCD19 CAR-T cells did not. These results were confirmed in a gadolinium uptake study.

B. Protective Agents

As set forth in this disclosure, some neurovascular pericytes and/or vSMCs express CD19 and become unintended targets of CD19-targeted therapy (on target, off tumor activity), which in turn leads to BBB damage and neurotoxic adverse events. One solution to this problem is to mask or block CD19 on pericytes and/or vSMCs so that it is not recognized by a CD19-targeted therapy, or so that binding of a CD19-targeted receptor (such as a CD19-targeted CAR) is reduced or prevented. Another solution is to down-regulate CD19 expression in pericytes and/or vSMCs, so that surface CD19 levels are reduced and the CD19⁺ pericytes and/or CD19+ vSMCs are affected less by CD19-targeted therapy. Another solution is to administer a bispecific binding agent that binds to a protein present on the neurovascular pericyte and/or vSMC surface, and activates an immunosuppressive mechanism in the CD19-targeted therapy, such as an immune checkpoint.

1. Protein Binding Agents

In one aspect, provided herein are protective agents capable of masking expression of CD19 by pericytes and/or vSMCs before administering a CD19-targeted therapy, such as a CD19 CAR-T cell or a BiTE® specific for CD19 and CD3. In some embodiments, provided herein are methods of reducing the neurotoxicity of a CD19-targeted therapy in a subject by intrathecal administration of a CD19 binding agent or a composition comprising a CD19 binding agent to the subject in connection with administration of the CD19-targeted therapy. Intrathecal administration makes these agents available to neurovascular pericytes and/or vSMCs, without providing similar protection to target CD19⁺ cells outside the central nervous system. The CD19 binding agent can be administered prior to administration of the CD19-targeted therapy, for example, allowing sufficient time for the CD19 binding agent to reach and bind to neurovascular pericytes and/or vSMCs. The neurotoxic symptoms of CD19-targeted therapy are not observed in every subject, and may not appear immediately (T. Jain et al., Blood Adv (2018) 2(22):3393-403). Thus, in some embodiments, the CD19 binding agent is administered at or near the time of administering CD19-targeted therapy, or after administering CD19-targeted therapy, for example, following observation of neurotoxic symptoms.

One method of treatment is to mask CD19 expressed on pericytes and/or vSMCs in connection with administering CD19-targeted therapy (such as a CAR-T or BiTE® specific for CD19). This can be accomplished by administering a CD19-binding agent that binds to CD19 and reduces binding by the CD19-targeted therapy, such as a specific antibody or antibody derivative. Numerous anti-CD19 antibodies and derivatives that can be used as CD19 binding agents are known in the art, for example, Coltuximabravtansine, SAR3419, SGN-CD19A, MOR208, MEDI-551, Denintuzumab mafodotin, DI-B4, Taplitumomabpaptox, XmAb 5871, MDX-1342, AFM11 (F. Naddafi et al., Int J Mol Cell Med (2015) 4(3):143-51). In some embodiments, the CD19 binding agent is an anti-CD19 antibody or an anti-CD19 antibody derivative that retains the ability specifically to bind CD19. Since the object of this method is to preserve CD19⁺ pericytes and/or CD19⁺ vSMCs, the antibody or derivative does not require cytotoxic activity, and antibodies and derivatives lacking an Fc portion or conjugated drug can be used. In some embodiments, the CD19 binding agent is an antibody that is not cytotoxic to CD19⁺ neurovascular pericytes and/or CD19⁺ vSMCs. Non-cytotoxic anti-CD19 antibodies are commercially available, and are used for applications such as fluorescence-activated cell sorting (FACS) where it is desirable to label a B cell without damaging it. Exemplary non-cytotoxic anti-CD19 antibodies include, without limitation, antibody HIB19 (Stemcell Technologies, cat #60005), CB19 (Novus Biologicals, cat #NBP2-26646), EPR5906 (abcam, cat #ab134114), clone SJ25-C1, Fab′2 (LSBio, cat #LS-C351479), IgG1 Clone #17 (Sino Biological, cat #11880-MM17), orb394446 (Biorbyt, cat #orb394446), 188-10263-1 (RayBiotech, cat #188-10263-1), and the like.

In some embodiments, the CD binding agent is an antibody derivative. In some embodiments, the antibody derivative is an scFv, a nanobody, an Fab′, or an F(ab′)₂. In some embodiments, the CD19 binding agent competes for CD19 binding with the CD19-targeted therapy. In some embodiments, the CD19 binding agent reduces the binding of the CD19-targeted therapy. In some embodiments, the CD19 binding agent and the CD19-targeted therapy are specific for the same CD19 epitope. In some embodiments, the CD19 binding agent and the CD19-targeted therapy are specific for overlapping epitopes. In some embodiments, the CD19 binding agent can bind reversibly or irreversibly.

Derivatives of CD19-specific antibodies are molecules that resemble antibodies in their mechanism of ligand binding, and include, for example, nanobodies, duobodies, diabodies, triabodies, minibodies, F(ab′)₂ fragments, Fab fragments, single chain variable fragments (scFv), single domain antibodies (sdAb), and functional fragments thereof. See for example, D. L. Porter et al., N Engl J Med (2011) 365(8):725-33 (scFv); E. L. Smith et al., Mol Ther (2018) 26(6):1447-56 (scFv); S. R. Banihashemi et al., Iran J Basic Med Sci (2018) 21(5):455-64 (CD19 nanobody); F. Rahbarizadeh et al., Adv Drug Deliv Rev (2019) 141:41-46 (sdAb); S. M. Kipriyanov et al., Int J Cancer (1998) 77(5):763-72 (diabody); F. Le Gall et al., FEBS Lett (1999) 453(1-2):164-68 (triabody); M. A. Ghetie et al., Blood (1994) 83(5):1329-36 (F(ab′)₂); and M.bA. Ghetie et al., Clin Cancer Res (1999) 5(12):3920-27 (F(ab′)₂ and Fab′). CD19-specific antibody derivatives can also be prepared from therapeutic anti-CD19 antibodies, for example without limitation, by preparing a nanobody, duobody, diabody, triabody, minibody, F(ab′)₂ fragment, Fab fragment, single chain variable fragment (scFv), or single domain antibody (sdAb) based on a therapeutic anti-CD19 antibody. Antibody derivatives can also be designed using phage display techniques (see, e.g., E. Romao et al., Curr Pharm Des (2016) 22(43):6500-18). In some embodiments, a CD19-specific antibody derivative is selected to bind to the same epitope as the CD19-targeted therapy. In some embodiments, a CD19-specific antibody derivative is derived from an antibody used in the CD19-targeted therapy. In some embodiments, for example in a CD19-targeted CAR-T therapy, the antigen-binding portion of the CAR is a CD19-specific scFv or nanobody. In some embodiments, the CD19-specific scFv or nanobody is also used as the protective agent. In some embodiments, the CD19 binding agent is a multi-specific binding agent that binds CD19 and one or more other pericyte and/or vSMC proteins.

Protective binding agents can also be multi-specific, for example a bispecific antibody or duobody, wherein the agent is specific for CD19 and another neurovascular pericyte and/or neurovascular vSMC surface protein, such as, for example without limitation, BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

Antibodies and antibody derivatives can also be administered in the form of a nucleic acid expression vector, for intracerebral, intrathecal, or intranasal administration and in situ generation of protective anti-CD19 antibodies or antibody derivatives. See, for example, K. Muthumani et al., Hum Vaccin Immunother (2013) 9(10):2253-62 (administration of AAV encoding Fab); K. Hollevoet et al., J Transl Med (2017) 15:131 (administration of antibodies and derivatives as a viral vector, DNA minicircle, naked DNA, or transcribed mRNA).

Nucleic acid expression vectors include, without limitation, plasmids, minicircles, viral vectors such as AAV, HSV, and lentiviral vectors, and the like.

In some embodiments, the expression vector can be a viral vector. The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

Nucleic acid sequences encoding the CD19-binding agents can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the binding agent disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the binding agents disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, viral vector, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences have been linked in a functionally operative manner, i.e., operably linked.

Also provided herein are vectors, plasmids, or viruses containing one or more nucleic acid molecules encoding any protective agent disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, J. Sambrook & D. W. Russell (2012). Molecular Cloning: A Laboratory Manual (4th ed.). Cold Spring Harbor, N.Y.: Cold Spring Harbor Laboratory and J. Sambrook & D. W. Russell (2001). Molecular Cloning: A Laboratory Manual (3rd ed.). Cold Spring Harbor, N.Y.: Cold Spring Harbor Laboratory (jointly referred to herein as “Sambrook”); F. M. Ausubel (1987). Current Protocols in Molecular Biology. New York, N.Y.: Wiley (including supplements through 2014); D. M. Bollag et al. (1996). Protein Methods. New York, N.Y.: Wiley-Liss; L. Huang et al. (2005) Nonviral Vectors for Gene Therapy. San Diego: Academic Press; M. G. Kaplitt et al. (1995) Viral Vectors: Gene Therapy and Neuroscience Applications. San Diego, Calif.: Academic Press; I. Lefkovits (1997) The Immunology Methods Manual: The Comprehensive Sourcebook of Techniques. San Diego, Calif.: Academic Press; Doyle, A. et al. (1998) Cell and Tissue Culture: Laboratory Procedures in Biotechnology. New York, N.Y.: Wiley; K. B. Mullis et al., (1994) PCR: The Polymerase Chain Reaction. Boston: Birkhauser Publisher; Greenfield, E. A. (2014) Antibodies: A Laboratory Manual (2nd ed.), New York, N.Y.: Cold Spring Harbor Laboratory Press; S. L. Beaucage et al., (2000) Current Protocols in Nucleic Acid Chemistry. New York, N.Y.: Wiley (including supplements through 2014); and S. C. Makrides (2003), Gene Transfer and Expression in Mammalian Cells. Amsterdam, N L: Elsevier Sciences B.V., the disclosures of which are incorporated herein by reference.

Viral vectors that can be used in the disclosure include, for example, retrovirus vectors (including lentivirus vectors), adenovirus vectors, and adeno-associated virus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In some embodiments, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some embodiments, the nucleic acid molecule is present in the recombinant host cell as a mini-circle expression vector for stable or transient expression.

The nucleic acid molecules can be encapsulated in a viral capsid or a lipid nanoparticle. Alternatively, endonuclease polypeptide(s) can be delivered by viral or non-viral delivery vehicles known in the art, such as electroporation or lipid nanoparticles. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also useful for nucleic acid delivery and gene therapy via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

The treatment disclosed herein can be used in combination with a CD19-targeted therapy for leukemia, lymphoma, or other CD19⁺ cancer. Indeed, there is an unmet medical need for means to reduce the cytotoxic effect of CD19-specific CAR-T cells on healthy, non-cancerous CD 19⁺ pericytes and/or CD19⁺ vSMCs without substantially diminishing the effect on CD19⁺ malignant cells. In one aspect, provided herein are methods of preventing neurotoxicity associated with on-target, off-tumor activity of CD19-specific CAR-T cells. In some embodiments, the method comprises a step of administering an effective amount of a CD19 binding agent specific for neurovascular pericytes and/or vSMCs without substantially binding to B cells or B lineage hyperproliferative cells to a subject in need thereof. In some embodiments, the CD19 binding agent specific for neurovascular pericytes and/or vSMCs is administered intrathecally. In some embodiments, the CD19 binding agent comprises a CD19-specific antibody or antibody derivative retaining the ability specifically to bind to CD19. In some embodiments, the CD19-specific antibody or antibody derivative retaining the ability specifically to bind to CD19 is an scFv or a nanobody specific for CD19. In some embodiments, the CD19 binding agent is administered as a nucleic acid that encodes the CD19-binding agent. In some embodiments, the nucleic acid encoding a CD19 binding protein is a plasmid, a viral vector, a minicircle, an mRNA, or a modified nucleic acid. In some embodiments, the CD19 binding agent is administered as a formulation comprising a pericyte-sparing and/or vSMC-sparing amount of the CD19 binding agent and a pharmaceutically acceptable carrier suitable for intrathecal administration. In some embodiments, the method of administering an effective amount of a CD19 binding agent to neurovascular pericytes and/or vSMCs in a subject in need thereof is by intrathecal administration. In some embodiments, the CD19 binding agent is an antibody or an antibody derivative. In some embodiments, the CD19 binding agent is an scFv or a nanobody specific for CD19. In some embodiments, the CD19 binding agent is administered as a nucleic acid that encodes a CD19 binding protein. In some embodiments, the nucleic acid is a plasmid, a viral vector, a minicircle, an mRNA, or a modified nucleic acid. In some embodiments, the CD19 binding agent is administered in a formulation comprising a protective amount of a CD19 binding agent and a carrier suitable for intrathecal administration.

In some embodiments, the antibody derivative is a nanobody, duobody, diabody, triabody, minibody, F(ab′)₂ fragment, Fab fragment, single chain variable fragment (scFv), or a single domain antibody (sdAb). In some embodiments, the antibody derivative that binds to CD19 comprises a CD19-specific nanobody or scFv. In some embodiments, the CD19 binding agent binds to CD19 expressed on neurovascular pericytes and/or vSMCs, and reduces neurotoxicity of a CD19-targeted chimeric antigen receptor T cell (CD19-targeted CAR-T cell) binding a CD19⁺ neurovascular pericyte and/or CD19⁺ vSMCs.

2. Expression Modulating Agents

In one aspect, provided herein are protective agents capable of reducing, inhibiting, or down-regulating expression of CD19 by pericytes and/or vSMCs. Inhibition of CD19 expression can be accomplished, for example, by using a nucleic acid or analog thereof that inhibits transcription or translation of CD19, such as, for example, an antisense oligonucleotide (ASO), or RNAi such as a small interfering RNA (siRNA), a short hairpin RNA (shRNA), and others. CD19 inhibitory oligonucleotides are known in the art: see, for example, N. Ishiura et al., Eur J Immunol (2010) 40:1192-204 (siRNA); S. Deaglio et al., Blood (2007) 109:5390-98 (siRNA); K. Wang et al., Exp Hematol Oncol (2012) 1:36; L. von Muenchow et al., Immunol Lett (2014) 160(2): 113-19; and are available commercially, e.g., from Millipore/Sigma, ThermoFisher Scientific, and Santa Cruz Biotechnology. In some embodiments, provided herein are methods of reducing the neurotoxicity of a CD19-targeted therapy in a subject by intrathecal administration of a CD19 inhibitor or a composition comprising a CD19 inhibitor to the subject at or before administration of the CD19-targeted therapy. In some embodiments, the CD19 inhibitor is selected from the group consisting of an ASO, an siRNA, and an shRNA.

In some embodiments, the CD19 inhibitor is an ASO capable of modulating the expression of CD19 by inhibiting or down-regulating it. Such modulation can produce an inhibition or reduction of expression of at least 20% compared to the normal expression level of CD19 in neurovascular pericytes and/or vSMCs, or at least 30%, 40%, 50%, 60%, 70%, 80%, or 90% inhibition compared to the normal expression level of CD19 in neurovascular pericytes and/or vSMCs. The target modulation is triggered by hybridization between a contiguous nucleotide sequence of the oligonucleotide and the CD19 nucleic acid. Expression can be determined in vivo or in vitro using immunohistochemistry specific for CD19.

In some embodiments, the ASO comprises a contiguous nucleotide sequence of 10 to 30 nucleotides in length, wherein the contiguous nucleotide sequence has at least about 90% sequence identity to CD19 and wherein the ASO is capable of reducing expression of CD19. In some embodiments, the ASO comprises one or more bases and/or linkages that do not occur naturally in DNA or RNA, such as phosphoramidite linkages, 2′-modified ribose or deoxyribose, morpholino phosphoramidites, peptide-nucleic acid links, locked nucleic acid links, xanthine, 7-methylguanine, inosine, dihydrouracil, 5-methylcytosine, 5-hydroxymethylcytosine, and others. Multiple ASOs having different sequences, or recognizing different segments of the CD19 sequence, can be used. See, e.g., C. I. E. Smith et al., Ann Rev Pharmacol Toxicol (2019) 59:605-30, incorporated herein by reference.

Nucleic acid-based CD19 inhibitors can also be administered as a vector that provides for expression of the inhibitor, such as a vector having a promoter operably linked to a nucleic acid that encodes an siRNA or shRNA, and is capable of expression thereof. Such vectors may take the form of plasmids, adenovirus vectors, adeno-associated virus (AAV) vectors, lentiviral vectors, and the like, as described above. In some embodiments, the vector is a viral vector. In some embodiments, the promoter in the vector is selected for preferential expression in pericytes and/or vSMCs. For example, the promoter may be derived from a protein that is characteristic of pericytes and/or vSMCs, such as PDGFRB, FOXF2, RGS5, or CD248, to reduce the chance that the vector will be expressed in cells that are the intended target of the CD19-targeted therapy. In some embodiments, the vector expresses a CD19 expression inhibitor. In some embodiments, a protective composition for reducing the neurotoxicity of a CD19-targeted therapy is provided, which composition comprises an effective amount of a vector that expresses a CD19 expression inhibitor, and a carrier suitable for intrathecal administration.

In one aspect, an expression modulating agent that down-regulates expression of CD19 in a neurovascular pericyte or vSMC is provided. In some embodiments, the expression modulating agent that comprises an antisense oligonucleotide (ASO), an siRNA, or a shRNA. In some embodiments, the expression modulating agent comprises an siRNA. In some embodiments, the expression modulating agent comprises an shRNA. In some embodiments, the expression modulating agent comprises an ASO.

3. Bispecific Binding Agents

In another aspect, bispecific binding agents as described herein are used in pericyte-sparing and/or vSMC-sparing methods and therapies. These bispecific binding agents have a first domain that binds to a T cell surface protein without activating the T cell, and a second domain that binds to a non-CD19 pericyte surface protein and/or to a non-CD19 vSMC surface protein. In an embodiment, the first domain binds to and activates an immune checkpoint surface protein on the T cell surface. This bispecific binding agent is designed to bind to pericytes and/or vSMCs, and to inhibit or disarm cytotoxic T cell action against the protected pericytes and/or vSMCs by activating an immune checkpoint in the T cell or inhibiting its response to antigen binding. This protective agent can be used in conjunction with a cell-based CD19-targeted therapy, for example with a CAR-T specific for CD19, or with any other therapy that recruits T cells or other cytotoxic cells to CD19⁺ cells, for example using a CD19/CD3 BiTE®. Bispecific binding agents of the disclosure reduce the neurotoxicity of CD19-targeted cell-based therapy on CD19⁺ neurovascular pericytes and/or CD19⁺ vSMCs as described herein.

Bispecific T cell-recruiting agents (for example BiTE®s) work by recruiting endogenous T cells to a selected target by binding an antigen on the target cell, and binding CD3 on a T cell, resulting in activation of the T cell and cytolysis of the target. Thus, a CD3/CD19-targeted BiTE® such as blinatumomab also acts like a cell-based CD19-targeted therapy for purposes of this disclosure, and can be rendered safer by using bispecific agents of the disclosure.

The degree to which neurotoxicity is reduced can be determined, for example, by measuring the number of pericytes and/or vSMCs killed or incapacitated by the CD19-targeted therapy with or without the bispecific binding agent, or by measuring the disruption of the BBB using, for example, the method set forth in Example 2 below.

Each binding domain can independently be, for example without limitation, an antibody, an antibody derivative such as an scFv or nanobody, a soluble receptor, a ligand or the interaction domain of a ligand, or other molecule capable of binding to the selected target. For example, one binding domain may be an scFv, while the other binding domain is a solubilized PD-L1 molecule or a PD-1 activating molecule. Either or both binding domains may be polyvalent, for example, having multiple binding sites for pericyte surface proteins and/or vSMC surface proteins, or multiple binding sites for immune checkpoint proteins. In some embodiments, the binding domain is an antibody, a nanobody, a diabody, a triabody, or a minibody, a F(ab′)₂ fragment, a Fab fragment, a single chain variable fragment (scFv), a single domain antibody (sdAb), or other functional antibody fragment. In some embodiments, the antigen-binding moiety includes a scFv. In some embodiments, the bispecific binding agent is a diabody, a triabody, a bispecific F(ab′)₂ fragment, or a bispecific antibody derivative.

The bispecific binding agent of the disclosure is designed to bind to both a pericyte and to an inhibitory surface protein, such as an immune checkpoint protein, on the CD19-targeted therapy cell. In some embodiments, the bispecific abinding agent of the disclosure is designed to bind to both a vSMC and to an inhibitory surface protein, such as an immune checkpoint protein, on the CD19-targeted therapy cell. The bispecific binding agent is designed to activate the inhibitory surface protein upon contact, thus reducing the CD19-targeted therapy cytotoxicity against neurovascular pericytes and/or vSMCs and preventing adverse effects on the BBB.

Multiple bispecific binding agents having different pairs of binding specificities can be employed, particularly when the agent targets multiple second targets, such as for example, two T cell surface proteins that provide an inhibitory effect or an increased inhibitory effect when the proteins are cross-linked with each other.

The first target of the bispecific binding agent is an immune checkpoint surface protein that inhibits the CD19-targeted therapy activity, for example, CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3 in the case of CD19-targeted therapy T cells. For purposes of the methods and binding agents herein, immune checkpoints include any surface protein that can be activated to reduce or eliminate T cell cytotoxicity. As these agents are designed so that they can be used in combination with CAR-T therapies (without limitation to such therapies), CAR-T cells can be provided with an inhibitory receptor which can be targeted by the bispecific binding agent (see, e.g., S. Sun et al., J Immunol Res (2018) ID:2386187). In some embodiments, the binding domain is specific and activates CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3. In some embodiments, the binding domain comprises an agonistic antibody, antibody derivative, nanobody, or scFv specific for CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3.

TIM-3 (T cell immunoglobulin and mucin-domain containing-3, also known as hepatitis A virus cellular receptor 2, HAVCR2) is an immune checkpoint protein expressed by activated T cells. Activation of TIM-3 by galectin-9 suppresses immune function in the T cell, and can cause apoptosis. Accordingly, the binding domain can comprise a non-antibody binding partner, such as galectin-9 or a TIM-3-binding fragment of galectin-9, or an antibody or antibody derivative that binds and activates TIM-3, or combinations thereof. In some embodiments, the binding agent is specific for and activates TIM-3. In some embodiments, the binding domain comprises galectin-9, a TIM-3-binding fragment of galectin-9, or an antibody or antibody derivative that binds and activates TIM-3.

PD-1 (programmed cell death protein 1) is an immune checkpoint protein expressed by activated T cells, B-cells, NK cells, and macrophages. Activation of PD-1 results in reduced T cell proliferation, cytokine release, and resistance to apoptosis (R. V. Parry et al., Mol Cell Biol (2005) 25(21)9543-53). In CD19-targeted therapy in which PD-1 expression is not downregulated or abrogated, a bispecific binding agent that binds and activates PD-1 can effectively reduce or eliminate cytotoxic action against pericytes and/or vSMCs. Bispecific binding agents that target and activate PD-1 can be prepared using, for example, agonistic antibodies (see, e.g., F. Bennett et al., J Immunol (2003) 170(2):711-18), PD-L1, PD-L2, or PD-1-binding fragments thereof (see, e.g., R. Li et al, Diabetes (2015) 64(2):529-40). In some embodiments, the binding agent is specific for and activates PD-1 or PD-2. In some embodiments, the binding domain comprises an antibody specific for PD-1, an antibody derivative specific for PD-1, a nanobody specific for PD-1, an scFv specific for PD-1, or a PD-1 binding domain derived from PD-L1 or PD-L2.

CTLA-4 (cytotoxic T lymphocyte antigen 4) is another immune checkpoint protein expressed by activated T cells, which acts as a negative regulator of T cell function. A bispecific binding agent that targets and activates CTLA-4 can be prepared using, for example, antibodies or derivatives that specifically bind CTLA-4 (S-J Shieh et al., J Immunol (2009) 183:2277-85), B7 fragments and analogs (W. Khamri et al., Gastroenterol (2017) 153:263-76), and other molecules that bind CTLA-4. In some embodiments, the binding agent is specific for and activates CTLA-4. In some embodiments, the binding domain comprises an antibody specific for CTLA-4, an antibody derivative specific for CTLA-4, a nanobody specific for CTLA-4, or an scFv specific for CTLA-4.

The adenosine A_{2A} receptor (A2AR, ADORA2, RDC8) is a G protein-coupled receptor found on immune cells and cardiac cells. In immune cells, the receptor acts to suppress immune function in the presence of extracellular adenosine. In addition to agonistic antibodies and antibody derivatives, a number of small molecule agonists are known, including ATL-146e; YT-146 (2-(1-octynyl)adenosine); CGS-21680; N6-(2-(3,5-dimethoxyphenyl)-2-(2-methylphenyl)-ethyl)adenosine; regadenoson; UK-432,097; limonene; zeatin riboside; 5′-(N-ethylcarboxamido)-adenosine); CV-3146; and biodenoson. In some embodiments, the binding agent is specific for and activates A2AR. In some embodiments, the binding domain comprises an antibody specific for A2AR, an antibody derivative specific for A2AR, a nanobody specific for A2AR, an scFv specific for A2AR, ATL-146e; 2-(1-octynyl)adenosine, CGS-21680, N⁶-(2-(3,5-dimethoxy-phenyl)-2-(2-methylphenylethyladenosine, regadenoson, UK-432,097, limonene, zeatin riboside, 5′-(N-ethylcarboxamido)adenosine, CV-3146, or biodenoson.

The second target of the bispecific binding agent is selected to bind a pericyte surface protein that distinguishes the pericyte from other cells that express CD19, such as B-lineage cells and malignant B-cells, but does not need to be unique to pericytes. In some embodiments, the second target of the bispecific binding agent is selected to bind a vSMC surface protein that distinguishes the vSMC from other cells that express CD19, such as B-lineage cells and malignant B-cells, but does not need to be unique to vSMCs. For example, the non-CD19 pericyte and/or non-CD19 vSMC surface protein can be BGN, FN1, SEMASA, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3, which are not known to be expressed on B-cells, and may not be expressed on malignant B-cells. Malignant tissue may be tested (e.g., through biopsy, liquid biopsy, radio-imaging with a labeled antibody, and the like) prior to initiating therapy in order to determine whether any pericyte and/or vSMC surface protein is already expressed by the target cells. In some embodiments, a binding domain comprises an antibody, an antibody derivative, a nanobody, or an scFv specific for BGN, FN1, SEMASA, CD248, PDGFR-β, CD146, RGSS, NG2, αSMA, desmin, PLXDC1, THY1, CDH6, COL1A2, ITGA1, EDNRA, CSPG4, AXL, NTM, TNFRSF1A, S1PR3, or F3. In some embodiments, a binding domain comprises an antibody, an antibody derivative, a nanobody, or an scFv specific for BGN, FN1, SEMA5A, CD146, PLXDC1, THY1, CDH6, COL1A2, ITGA1, EDNRA, CSPG4, AXL, NTM, TNFRSF1A, S1PR3, or F3.

CD248 (endosialin) is a Group XIV C-type lectin family member expressed on the surface of activated mesenchymal cells, and reported to be dynamically expressed by pericytes and vSMCs and fibroblasts during tissue development, tumor neovascularization, and inflammation. It has also been associated with stromal cell proliferation and migration, and has been suggested as a biomarker for sarcoma and related disorders. See, e.g., B. A. Teicher, Oncotarget (2019) 10(9):993-1009. In some embodiments, the second binding domain is specific for CD248. In some embodiments, the binding domain comprises an antibody specific for CD248, an antibody derivative specific for CD248, a nanobody specific for CD248, or an scFv specific for CD248.

RGSS (regulator of G-protein signaling 5) is a tissue-specific signal regulation protein associated with vasculature development. The highest expression appears in pericytes and vascular smooth muscle cells (vSMCs), in the brain and cardiovascular tissue. RGSS is highly expressed in some solid tumors, and is implicated in angiogenesis pathways. See, e.g., K. J. Perschbacher et al., Physiol Genomics (2018) 50:590-604. In some embodiments, the second binding domain is specific for RGS5. In some embodiments, the binding domain comprises an antibody specific for RGS5, an antibody derivative specific for RGS5, a nanobody specific for RGS5, or an scFv specific for RGS5.

PLXDC1 (plexin domain containing 1) plays a role in endothelial cell capillary morphogenesis, and is expressed on the surface of CD19⁺ neurovascular pericytes and/or CD19+ vSMCs. In some embodiments, the second binding domain is specific for PLXDC1. In some embodiments, the binding domain comprises an antibody specific for PLXDC1, an antibody derivative specific for PLXDC1, a nanobody specific for PLXDC1, or an scFv specific for PLXDC1.

CDH6 and CDH11 (cadherin 6 and cadherin 11) are calcium-dependent cell adhesion proteins expressed on the surface of CD19⁺ neurovascular pericytes and/or CD19⁺ vSMCs. In some embodiments, the second binding domain is specific for CDH6 or CDH11. In some embodiments, the binding domain comprises an antibody specific for CDH6 or CDH11, an antibody derivative specific for CDH6 or CDH11, a nanobody specific for CDH6 or CDH11, or an scFv specific for CDH6 or CDH11.

TFPI (tissue factor pathway inhibitor) inhibits factor X, inhibits VIIa/tissue factor activity, thrombotic action, and the ability to associate with lipoproteins in plasma. It is expressed on the surface of CD19⁺ neurovascular pericytes and/or CD19⁺ vSMCs. In some embodiments, the second binding domain is specific for TFPI. In some embodiments, the binding domain comprises an antibody specific for TFPI, an antibody derivative specific for TFPI, a nanobody specific for TFPI, or an scFv specific for TFPI.

THY1 (THY1 surface antigen) is thought to play a role in cell-cell or cell-ligand interactions during synaptogenesis and other events in the brain, and is expressed on the surface of CD19⁺ neurovascular pericytes and/or CD19⁺ vSMCs. In some embodiments, the second binding domain is specific for THY1. In some embodiments, the binding domain comprises an antibody specific for THY1, an antibody derivative specific for THY1, a nanobody specific for THY1, or an scFv specific for THY1.

ITGA1 (integrin α1 subunit) forms a receptor for laminin and collagen, and is involved in anchorage-dependent, negative regulation of EGF-stimulated cell growth, and is expressed on the surface of CD19⁺ neurovascular pericytes and/or CD19⁺ vSMCs. In some embodiments, the second binding domain is specific for ITGA1. In some embodiments, the binding domain comprises an antibody specific for ITGA1, an antibody derivative specific for ITGA1, a nanobody specific for ITGA1, or an scFv specific for ITGA1.

COL1A2 (collagen type I α2 chain) is a collagen protein expressed on the surface of CD19⁺ neurovascular pericytes and/or CD19⁺ vSMCs. In some embodiments, the second binding domain is specific for COL1A2. In some embodiments, the binding domain comprises an antibody specific for COL1A2, an antibody derivative specific for COL1A2, a nanobody specific for COL1A2, or an scFv specific for COL1A2.

EDNRA (endothelin receptor type A) is expressed on the surface of CD19+ neurovascular pericytes and/or CD19⁺ vSMCs. In some embodiments, the second binding domain is specific for EDNRA. In some embodiments, the binding domain comprises an antibody specific for EDNRA, an antibody derivative specific for EDNRA, a nanobody specific for EDNRA, or an scFv specific for EDNRA.

PCDH18 (protocadherin 18) is a cadherin-related neuronal receptor thought to play a role in the establishment and function of specific cell-cell connections in the brain, and is expressed on the surface of CD19⁺ neurovascular pericytes and/or CD19+ vSMCs. In some embodiments, the second binding domain is specific for PCDH18. In some embodiments, the binding domain comprises an antibody specific for PCDH18, an antibody derivative specific for PCDH18, a nanobody specific for PCDH18, or an scFv specific for PCDH18.

AXL (AXL receptor tyrosine kinase) is a surface receptor for GAS6, and is expressed on the surface of CD19⁺ neurovascular pericytes and/or CD19⁺ vSMCs. In some embodiments, the second binding domain is specific for AXL. In some embodiments, the binding domain comprises an antibody specific for AXL, an antibody derivative specific for AXL, a nanobody specific for AXL, or an scFv specific for AXL.

NTM (neurotrimin) is a neural cell adhesion molecule expressed on the surface of CD19⁺ neurovascular pericytes and/or CD19⁺ vSMCs. In some embodiments, the second binding domain is specific for NTM. In some embodiments, the binding domain comprises an antibody specific for NTM, an antibody derivative specific for NTM, a nanobody specific for NTM, or an scFv specific for NTM.

TNFRSF1A (tumor necrosis factor receptor superfamily member 1A) is a receptor for TNFSF2/TNFα and homotrimeric TNFSF1/lymphotoxin-α, and is expressed on the surface of CD19⁺ neurovascular pericytes and/or CD19⁺ vSMCs. In some embodiments, the second binding domain is specific for TNFRSF1A. In some embodiments, the binding domain comprises an antibody specific for TNFRSF1A, an antibody derivative specific for TNFRSF1A, a nanobody specific for TNFRSF1A, or an scFv specific for TNFRSF1A.

S1PR3 (sphingosine-1-phosphate receptor) is a receptor for the lysosphingolipid sphingosine 1-phosphate (S1P), and is expressed on the surface of CD19⁺ neurovascular pericytes and/or CD19⁺ vSMCs. In some embodiments, the second binding domain is specific for S1PR3. In some embodiments, the binding domain comprises an antibody specific for S1PR3, an antibody derivative specific for S1PR3, a nanobody specific for S1PR3, or an scFv specific for S1PR3.

F3 (coagulation factor III, tissue factor) initiates blood coagulation by forming a complex with circulating factor VII or VIIa, and is expressed on the surface of CD19+ neurovascular pericytes and/or CD19⁺ vSMCs. In some embodiments, the second binding domain is specific for F3. In some embodiments, the binding domain comprises an antibody specific for F3, an antibody derivative specific for F3, a nanobody specific for F3, or an scFv specific for F3.

NG2 (neural/glial antigen 2, chondroitin sulfate proteoglycan 4, CSPG4) is an integral membrane proteoglycan found on pericytes and/or vSMCs, also known as high molecular weight melanoma-associated antigen. NG2 is not constitutively expressed in adult neurovascular pericytes and/or vSMCs, but is apparently expressed during CNS development, in pathological conditions, and upon CNS injury (G. Ferrara et al., Acta Neuropathol (2016) 132:23-42). See, e.g., X. Wang et al., Cancer Res (2011) 71(24):7410-22. In some embodiments, the binding domain is specific for NG2. In some embodiments, the binding domain comprises an antibody specific for NG2, an antibody derivative specific for NG2, a nanobody specific for NG2, or an scFv specific for NG2.

BGN (biglycan, also called proteoglycan-I, DSPG1, PG-S1, PGI, SLRR1A, SEMDX, and MRLS) is a small, leucine-rich repeat proteoglycan found in the extracellular matrix. The protein carries to glycosaminoglycan chains, composed of either chondroitin sulfate or dermatan sulfate. It is associated with fibroblasts, myofibroblasts, endothelial cells, pericytes, vSMCs, and macrophages (H. Alimohamad et al., J Periodontal Res (2005) 40(1):73-86). The core protein is highly conserved across species. In some embodiments, the second binding domain is specific for BGN. In some embodiments, the binding domain comprises an antibody specific for BGN, an antibody derivative specific for BGN, a nanobody specific for BGN, or an scFv specific for BGN.

FN1 (fibronectin) is a high molecular weight glycoprotein found in the extracellular matrix. FN1 binds to integrins, as well as collagen, fibrin, and heparin sulfate proteoglycans. In some embodiments, the binding domain is specific for FN1. In some embodiments, the second binding domain comprises an antibody specific for FN1, an antibody derivative specific for FN1, a nanobody specific for FN1, or an scFv specific for FN1.

SEMA5A (semaphorin-5A) is a transmembrane protein associated with angiogenesis and promotion of endothelial cell proliferation. Although associated primarily with guiding axons during development, SEMA5A has also been shown to induce TNFα and IL-8 (M. Sugimoto et al., Proc Natl Acad Sci USA (2006) 103(7):6454-59). In some embodiments, the second binding domain is specific for SEMA5A. In some embodiments, the binding domain comprises an antibody specific for SEMA5A, an antibody derivative specific for SEMA5A, a nanobody specific for SEMA5A, or an scFv specific for SEMA5A.

PDGFRB (platelet-derived growth factor receptor-β) is a receptor tyrosine kinase that is activated by PDGF. PDGFRB is activated during embryogenesis, and is associated with pericyte formation, and vascular smooth muscle cells (vSMCs). In some embodiments, the second binding domain is specific for PDGFRB. In some embodiments, the binding domain comprises an antibody specific for PDGFRB, an antibody derivative specific for PDGFRB, a nanobody specific for PDGFRB, or an scFv specific for PDGFRB.

CD146 (also known as MCAM, melanoma cell adhesion molecule, and cell surface glycoprotein MUC18) is a receptor for laminin-α4, and is highly expressed in vascular endothelial cells, smooth muscle cells (e.g., vSMCs), and pericytes. It acts as a co-receptor of PDGFRB and is required for development and maintenance of the blood-brain barrier (J. Chen et al., Proc Nall Acad Sci USA (2017) 114(36):E7622-31). In some embodiments, the second binding domain is specific for CD146. In some embodiments, the bispecific binding agent comprises an antibody specific for MCAM, an antibody derivative specific for MCAM, a nanobody specific for MCAM, or an scFv specific for MCAM.

Desmin (DES, CSM1, LGMD2R) is a muscle-specific type III intermediate filament that regulates sarcomere architecture. In some embodiments, the binding domain is specific for DES. In some embodiments, the binding domain comprises an antibody specific for DES, an antibody derivative specific for DES, a nanobody specific for DES, or an scFv specific for DES.

Smooth muscle actin-α (αSMA, ACTA2, MYMY5) is an actin protein involved in the contractile apparatus of smooth muscle (A. E. van der Wijk et al., Tissue Cell (2018) 52:42-50). In some embodiments, the binding domain is specific for αSMA. In some embodiments, the second binding domain comprises an antibody specific for αSMA, an antibody derivative specific for αSMA, a nanobody specific for αSMA, or an scFv specific for αSMA.

In some embodiments, the first target is BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3, or a combination thereof. In some embodiments, the first target is BGN, FN1, SEMA5A, or a combination thereof. In some embodiments, the first target is BGN or SEMA5A, or a combination thereof. In some embodiments, the first target is SEMA5A. In some embodiments, the first target is CD248, RGS5, NG2, or desmin, or a combination thereof. In some embodiments, the first target is CD248. In some embodiments, the target is NG2. In another embodiment of the disclosure, the bispecific binding agent second target is CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3. In some embodiments, the second binding domain is an antibody, antibody derivative, nanobody, or scFv that specifically binds and activates CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3. In some embodiments, the bispecific binding agent has one binding domain comprising an antibody, an antibody derivative, a nanobody, or an scFv specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3; and a second binding domain comprising an antibody, an antibody derivative, a nanobody, an scFv, or a binding partner specific and activating for CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3.

Strategies for the design and construction of therapeutic agents with two or more specificities are known in the art. See, e.g., S. Chen et al., J Immunol Res (2019) 2019:ID4516041; E. Hatterer et al., MAbs (2019) 11(2):322-34; M. Fu et al., Front Immunol (2019) 10:1396; R. Ahamadi-Fesharaki et al., Mol Ther Oncolytics (2019) 14:38-56; Y. Xu et al., MAbs (2015) 7(1):231-42.

Bispecific binding agents can also be administered in the form of a nucleic acid that encodes the bispecific binding agent, where the nucleic acid is expressed in the subject following administration. The nucleic acid can be contained within a vector, such as an expression vector having a promoter that is functional in a mammalian cell, and is operably linked to a nucleic acid that encodes the agent. The nucleic acid can be administered in the form of a virus containing the nucleic acid, or a cell transduced with the nucleic acid. See, e.g., U.S. Pat. No. 1,039,1132.

C. Methods and Systems for CD19-Targeted Therapy

As set forth above, CD19-targeted therapy such as anti-CD19 CAR-T and BiTE treatment can be very effective in the treatment of CD19⁺ B-cell hyperproliferative disorders such as ALL and CLL. Systems of the disclosure comprise a CD19-targeted therapy in combination with an agent that reduces the adverse impact of CD19-targeted therapy on CD19⁺ neurovascular pericytes and/or CD19⁺ vSMCs. Thus, in one aspect, provided herein are systems comprising a CD19-targeted therapy and a protective agent that reduces damage to the blood-brain barrier (BBB) that can be caused by administration of the CD19-targeted therapy. In some embodiments, the agent (a) binds to CD19 and reduces binding of the CD19-targeted therapy to CD19⁺ neurovascular pericytes and/or CD19⁺ vSMCs; (b) down-regulates expression of CD19 by neurovascular pericytes and/or vSMCs; or (c) comprises a bispecific binding agent that binds to a non-CD19 pericyte surface protein and/or non-CD19 vSMC surface protein and activates an immune checkpoint surface protein on the CD19-targeted therapy cell if it attempts to attack the pericyte. The systems provided herein reduce damage and disruption of the BBB by CD19-targeted therapy by protecting CD19⁺ neurovascular pericytes and/or CD19⁺ vSMCs from cytotoxic activity associated with the CD19-targeted therapy.

In some embodiments, the protective agent reduces the neurotoxicity of a CD19-targeted therapy by at least about 10%, 15%, 20%, 25%, 30%, 40%, 50%, 60%, 70%, 75%, 80%, 85%, 90%, 95%, 98%, or about 100%, as determined by the number of pericytes and/or vSMCs killed or incapacitated by the CD19-targeted therapy in the presence or absence of the CD19 binding agent. This can be quantified using immunohistochemistry, vital dye staining, and other methods known in the art, for example, in a suitable animal model. Reduction in neurotoxicity can also be measured by the reduction or elimination of symptoms in subjects receiving CD19-targeted therapy, for example, in a clinical trial. Reduction in neurotoxicity can also be measured by determining if damage has occurred to the BBB, for example by administering labeled (for example, radio-labeled) albumin to a subject receiving CD19-targeted therapy, followed by determination of whether or not the labeled albumin crosses the BBB. Alternatively, the degree of damage to the BBB can be estimated using serum biomarkers such as, for example without limitation, S100B (see, e.g., B. J. Blyth et al., J Neurotrauma (2009) 26(9):1497-507).

In some embodiments, the system comprises a CD19-targeted therapy and a protective CD19 binding agent. In some embodiments, the protective agent is an anti-CD19 antibody or an anti-CD19 antibody derivative. In some embodiments, the CD19 binding agent is an antibody that is not cytotoxic to CD19⁺ neurovascular pericytes and/or CD19⁺ vSMCs. In some embodiments, the CD binding agent is an antibody derivative. In some embodiments, the antibody derivative is an scFv, a nanobody, an Fab′, or an F(ab′)₂. In some embodiments, the CD19 binding agent competes for CD19 binding with the CD19-targeted therapy. In some embodiments, the CD19 binding agent reduces the binding of the CD19-targeted therapy. In some embodiments, the CD19 binding agent and the CD19-targeted therapy are specific for the same CD19 epitope. In some embodiments, the CD19 binding agent and the CD19-targeted therapy are specific for overlapping epitopes. In some embodiments, the CD19 binding agent can bind reversibly or irreversibly.

The reduction of neurotoxicity, and the reduction in disruption of or damage to the BBB, can alternatively be determined using an in vivo animal model, for example using a protein exclusion or dye exclusion model as set forth in Example 2 below. In some embodiments, the protective agent reduces neurotoxicity, and/or BBB damage, caused by a CD19-targeted therapy by at least about 10%, 15%, 20%, 25%, 30%, 40%, 50%, 60%, 70%, 75%, 80%, 85%, 90%, 95%, 98%, or about 100%, as determined by: (a) a comparison of the number of pericytes and/or vSMCs killed by a CD19-targeted therapy in the presence of the protective agent vs. the number of pericytes and/or vSMCs killed by the CD19-targeted therapy in the absence of the protective agent in vitro or in vivo; (b) a comparison of the neurological symptoms caused by a CD19-targeted therapy in the presence of the protective agent vs. the neurological symptoms caused by the CD19-targeted therapy in the absence of the protective agent in an animal model; or (c) a comparison of the disruption of the BBB caused by a CD19-targeted therapy in the presence of the protective agent vs. the disruption of the BBB caused by the CD19-targeted therapy in the absence of the protective agent in an animal model (which can be quantified by the testing ability of the BBB to exclude a labeled protein in the vascular system from brain or spinal tissue). In some embodiments, the disruption of the BBB caused by the CD19-targeted therapy is quantified by measuring the amount of serum albumin that passes the BBB and invades CNS tissue in a mouse. In some embodiments, the amount of serum albumin is quantified directly. In some embodiments, the amount of serum albumin is quantified using labeled serum albumin, for example radioactively labeled serum albumin In some embodiments, the serum albumin is labeled using a dye that binds to serum albumin In some embodiments, the dye is Evans Blue dye. In some embodiments, the presence of neurological symptoms and/or BBB disruption is taken as an indication that neurovascular pericytes and/or vSMCs have been killed or incapacitated.

In another aspect, provided herein are methods of aiding in the treatment of a CD19⁺ B-cell hyperproliferative disorder, comprising administering to a subject who is receiving, or will receive a CD19-targeted therapy, a protective agent that reduces damage to the BBB that can be caused by administration of the CD19-targeted therapy. In some embodiments, the protective agent: (a) binds to CD19 and reduces binding of the CD19-targeted therapy to CD19⁺ neurovascular pericytes and/or CD19⁺ vSMCs; (b) down-regulates expression of CD19 by neurovascular pericytes and/or vSMCs; or comprises a bispecific binding agent that binds to a non-CD19 pericyte surface protein and/or a non-CD19 vSMC surface protein and an immune checkpoint surface protein, wherein binding to the immune checkpoint protein reduces the CD19-targeted therapy neurotoxicity.

1. CD19-Targeted Therapy using CAR-T

CD19-targeted therapies of the disclosure include the use of cells that have been modified to express a chimeric antigen receptor (CAR) that is specific for CD19, and other therapies that target CD19 and recruit T cells, such as therapy using a CD3/CD19-targeted BiTE®.

In general, a CAR comprises an extracellular antigen binding domain, a transmembrane domain, and an intracellular signaling domain. The extracellular domain often also comprises a spacer or hinge sequence between the antigen binding domain and the transmembrane domain. Hinge sequences can be derived from the hinge region of proteins such as IgG, CD8A, CD28, and similar proteins. Alternatively, the hinge sequence can be a flexible synthetic linker, for example, without limitation, a glycine-serine polymer like (GGS)ₙ, (GSGG)ₙ, (GGGS)ₙ, and the like.

The CAR antigen binding domain can include any class of domain that binds to the antigen of interest (CD19) and may include, but is not limited to, a monoclonal antibody, a polyclonal antibody, a synthetic antibody, a human antibody, a humanized antibody, a non-human antibody, and any antigen-binding fragment thereof. In some embodiments, the antigen binding domain portion comprises a mammalian antibody or a fragment thereof. The antigen binding domain may comprise a commercial antibody or a fragment thereof that binds to a target antigen. In some embodiments, the CAR antigen binding domain comprises an scFv, nanobody, Fab, or (Fab′)₂ fragment, specific for CD19. In an embodiment, the CAR antigen binding domain comprises an scFv.

The transmembrane domain comprises a hydrophobic sequence that anchors the receptor in the plasma membrane. Commonly used transmembrane domains include, for example, the transmembrane domain from CD3 zeta (CD3ζ) or CD28. Additional examples of transmembrane domains include, without limitation, the transmembrane domains from CD3ε, CD45, CD4, CD5, CD7, CD8, CD9, CD16, CD22, CD33, CD37, CD64, CD80, CD86, CD134 (OX40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain is synthetic, and comprises predominantly hydrophobic residues such as leucine and valine. In some embodiments, a triplet of phenylalanine, tryptophan and valine is present at either or both ends of a synthetic transmembrane domain.

The CAR signaling domain comprises one or more signaling regions, often derived from the immunoreceptor tyrosine-based activation motifs (ITAMs) found in the cytoplasmic region of CD3ζ. The signaling domain can contain multiple copies of the ITAM, and can further comprise signaling domains from co-stimulatory receptors required for T cell activation, proliferation, and survival. Suitable co-stimulatory signaling domains include, for example, those derived from CD27, CD28, OX40 (CD134), and 4-1BB (CD137). In some embodiments, the CAR signaling domain comprises a CD3ζ ITAM. In an embodiment, the CAR signaling domain comprises a CD3ζ ITAM and a co-stimulatory signaling domain from one or more of CD27, CD28, OX40, and 4-1BB. In an embodiment, the CAR signaling domain comprises a CD3ζ ITAM and a co-stimulatory signaling domain from CD28 and 4-1BB.

Methods for administering immune cells for therapy, such as CD19-targeted therapies, are known and may be used in connection with the provided methods and compositions. For example, adoptive T cell therapy methods are described in US 2003/0170238; U.S. Pat. No. 4,690,915; S. A. Rosenberg, Nat Rev Clin Oncol (2011) 8(10):577-85. See also M. Themeli et al., Nat Biotechnol (2013) 31(10):928-33; and T. Tsukahara et al., Biochem Biophys Res Commun (2013) 438(1):84-89. In an aspect of the disclosure, the method comprises administering a CD19-targeted CAR-T cell in combination with a protective agent of the disclosure. In an aspect of the disclosure, the method comprises administering tisagenlecleucel or axicabtagene in combination with a protective agent of the disclosure. In another aspect of the disclosure, the method comprises administering a CD3/CD19-targeted BiTE® in combination with a protective agent of the disclosure. In another aspect of the disclosure, the method comprises administering blinatumomab in combination with a protective agent of the disclosure.

2. Protective Agents

The protective agents of the disclosure (e.g., protein binding agents, expression modifying agents, and bispecific binding agents) are administered in conjunction with a CD19-targeted therapy for the treatment of a CD19⁺ B-cell hyperproliferative disorder. In some embodiments, the protective agent is administered at or before the time of administration of the CD19-targeted therapy. In some embodiments, the protective agent is administered about 15, 10, 9, 8, 7, 6, 5, 4, 3, 2, or 1 days, or about 23, about 22, about 21, about 20, about 19, about 18, about 17, about 16, about 15, about 14, about 13, about 12, about 11, about 10, about 9, about 8, about 7, about 6, about 5, about 4, about 3, about 2, or about 1 hours before the first administration of the CD19-targeted therapy. In some embodiments, the protective agent is administered within about one hour before to about one hour after the beginning of the first administration of the CD19-targeted therapy. In some embodiments, the protective agent is administered throughout the CD19-targeted therapy treatment period. In some embodiments, the protective agent is administered continuously during the administration of the CD19-targeted therapy. In some embodiments, the protective agent is first administered about one hour after the first administration of the CD19-targeted therapy. In some embodiments, the protective agent is first administered about 2, 3, 4, 5, 6, 7, 8, 9, 10, 11, 12, 13, 14, 15, 16, 17, 18, 19, 20, 21, 22, 23, or 24 hours after the first administration of the CD19-targeted therapy. In some embodiments, the protective agent is first administered about 2, 3, 4, 5, 6, 7, 8, 9, 10, 11, or days after the first administration of the CD19-targeted therapy.

In some embodiments, the protective agent is first administered about one hour after the first appearance of symptoms consistent with neurotoxicity following administration of the CD19-targeted therapy. In some embodiments, the protective agent is first administered about 2, 3, 4, 5, 6, 7, 8, 9, 10, 11, 12, 13, 14, 15, 16, 17, 18, 19, 20, 21, 22, 23, or 24 hours after the first appearance of symptoms. Common symptoms of neurotoxicity can include headache, malaise, confusion, somnolence, disorientation, ataxia, seizure, aphasia, and stupor. Early signs may include tremors and agraphia (tested using a handwriting test). (T. Jain et al., Blood Adv (2018) 2(22):3393-403.)

Protective agents can be administered once, multiple times, continuously, or as needed, depending on the pharmacokinetics of the agent and the needs of the subject. In an embodiment, the protective agent is administered once, prior to administering a CD19-targeted therapy. In another embodiment, the protective agent is administered 2, 3, 4, 5, 6, 7, 8, 9, or 10 times prior to administering a CD19-targeted therapy. In another embodiment, the protective agent is administered at the time of administering a CD19-targeted therapy. In another embodiment, the protective agent is infused continuously at the time of administering a CD19-targeted therapy, and continuing after the CD19-targeted therapy has been administered. In another embodiment the protective agent is first administered as a bolus prior to administering a CD19-targeted therapy, then infused continuously at the time of administering a CD19-targeted therapy, and continuing after the CD19-targeted therapy has been administered.

Proteinaceous agents (such as protein-based binding agents and bispecific binding agents) can be administered in the same manner as a therapeutic antibody, and using similar formulations as are known in the art. Similarly, nucleic acid-based agents (such as ASOs, siRNAs, shRNAs, and other nucleic acids encoding protein binding agents and bispecific binding agents) can be administered using methods known for the administration of ASOs and viral or plasmid expression vectors. All agents can be administered parenterally. Bispecific binding agents can be administered in the same mode as the CD19-targeted therapy. Protein binding agents, nucleic acids encoding protein binding agents, and expression modulating agents can advantageously be administered directly to the CNS, for example by intranasal or intrathecal administration, or can be administered parenterally using a CNS-targeting formulation, such as, for example, a lipid nanoparticle, liposome, or dendrimer conjugated with a CNS-homing protein such as a viral envelope protein derived from rabies, pseudorabies, or a herpesvirus, as described below.

Intrathecal administration may comprise injection in the cervical region of the spinal canal, in the thoracic region of the spinal canal, or in the lumbar region of the spinal canal. Typically, intrathecal administration is performed by injecting an agent, for example a protective composition comprising a CD19 binding agent, into the subarachnoid cavity (subarachnoid space) of the spinal canal, between the arachnoid membrane and pia mater of the spinal canal.

One aspect of the disclosure is a method for aiding in the treatment of a CD19⁺ B-cell hyperproliferative disorder by administering a protective agent, and administering a CD19-targeted therapy. Another aspect of the disclosure is a method for treating a CD19⁺ B-cell hyperproliferative disorder by administering a protective agent, and administering a CD19-targeted therapy. In some embodiments, the protective agent is: (a) a protein binding agent that binds to CD19 on neurovascular pericytes and/or vSMCs and reduces binding of CD19-targeted therapy to CD19 on neurovascular pericytes and/or vSMCs; (b) an agent that down-regulates the expression of CD19 by neurovascular pericytes and/or vSMCs; or (c) a bispecific binding agent that binds to a non-CD19 pericyte surface protein and/or a non-CD19 vSMC surface protein and an immune checkpoint surface protein, wherein binding to the non-CD19 pericyte surface protein and/or the non-CD19 vSMC surface protein and the immune checkpoint protein reduces the CD19-targeted therapy neurotoxicity. In some embodiments, the protective agent comprises an antibody or an antibody derivative that is specific for CD19. In some embodiments, the antibody derivative is a nanobody or an scFv. In some embodiments, the agent of part (b) comprises an ASO, siRNA, or shRNA. In some embodiments, the agent of part (c) comprises a bispecific binding agent wherein one binding domain comprises an antibody, an antibody derivative, a nanobody, or an scFv specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3; and a second binding domain comprises an antibody, an antibody derivative, a nanobody, an scFv, or other binding moiety that specifically binds and activates CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3. In some embodiments, the method comprises administering a combination of two or more of the agents of (a), (b), and (c) above.

As malignant cells often evolve and change the surface proteins they express, it may be appropriate to switch the first target binding specificity from one pericyte surface protein and/or one vSMC surface protein to another periodically, to prevent the development of resistance. For example, if a bispecific binding agent that binds NG2 is selected first, a subpopulation of lymphoma cells that has mutated to also express NG2 would be advantaged compared to other lymphoma cells and, being protected from the CD19-targeted therapy could expand to replace the lymphoma cells that were successfully treated by the CD19-targeted therapy. Periodic switching of the binding target between different pericyte surface proteins and/or different vSMC surface protein can prevent or reduce such reactive resistance. Additionally, two or more bispecific binding agents, having different pericyte and/or vSMC targets, can be used simultaneously to reduce the probability of reactive resistance, as the probability of lymphoma cells having two protective mutations is lower than the probability of having only one protective mutation. In some embodiments, two bispecific binding agents, each of which binds a different pericyte and/or vSMC surface protein, are administered. In some embodiments, the two different bispecific binding agents are administered at the same time. In some embodiments, the two different bispecific binding agents are administered in succession. In some embodiments, more than two different bispecific binding agents are administered. In some embodiments, 3, 4, 5, 6, 7, 8, 9, or 10 different bispecific binding agents are administered.

In some embodiments, the CD19-targeted therapy comprises an anti-CD19 chimeric antigen receptor T cell (CAR-T) or a bispecific T cell engager specific for CD19. In some embodiments, the binding agent comprises an antibody or antibody derivative that binds to CD19. In some embodiments, the antibody derivative is a nanobody, duobody, diabody, triabody, minibody, F(ab')2 fragment, Fab fragment, single chain variable fragment (scFv), or a single domain antibody (sdAb). In some embodiments, the agent comprises a nanobody or an scFv.

In some embodiments, the agent is an expression modulator. In some embodiments, the expression modulator comprises an antisense oligonucleotide (ASO), an siRNA, or a shRNA. In some embodiments, the expression modulator comprises an antisense oligonucleotide (ASO), an siRNA, or a shRNA. In some embodiments, the expression modulator comprises an siRNA. In some embodiments, the expression modulator comprises an shRNA. In some embodiments, the expression modulator comprises an ASO.

In some embodiments, the agent is a bispecific binding agent that comprises a first binding domain having affinity for an immune checkpoint surface protein, and a second binding domain having affinity for a non-CD19 pericyte surface protein and/or a non-CD19 vSMC surface protein, wherein binding of the bispecific binding agent to both a pericyte and/or vSMC surface protein and agonizes an immune checkpoint surface protein, such as a checkpoint protein, causes inhibition of an immune cell activity. In some embodiments, the immune cell activity that is inhibited is cytotoxicity. In some embodiments, the immune checkpoint surface protein comprises CTLA-4, A2AR, VTCN1, BTLA, PD-1, or TIM-3. In some embodiments, the first binding domain comprises a CTLA-4-binding domain of CD80 or CD86. In some embodiments, the first binding domain comprises a CTLA-4 agonist. In some embodiments, the CTLA-4 agonist comprises an antibody or an antibody derivative. In some embodiments, the first binding domain comprises a PD-1 binding domain of PD-L1 or PD-L2, or an antibody or an antibody derivative specific for PD-1. In some embodiments, the first binding domain comprises a PD-1 agonist. In some embodiments, the first binding domain comprises an A2AR binding agent. In some embodiments, the A2AR binding agent comprises an antibody or a derivative thereof specific for A2AR. In some embodiments, the A2AR binding agent comprises an A2AR agonist.

In some embodiments, the non-CD19 pericyte surface protein and/or the non-CD19 vSMC surface protein comprises BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3. In some embodiments, the second domain comprises an antibody or a derivative thereof specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

D. Formulations

In some embodiments, the proteins and nucleic acids of the disclosure are incorporated into compositions, including pharmaceutical compositions. Such compositions typically include the nucleic acids, and/or proteins, and a pharmaceutically acceptable excipient or carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), and the like. In all cases, the composition should be sterile, and should be sufficiently fluid to administer by syringe. It should be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, for example, sodium dodecyl sulfate. Prevention of microorganism activity can be achieved by various antibacterial and antifungal agents, for example, parabens, chlorobutanol, phenol, ascorbic acid, thimerosal, and the like. In many cases, it is useful to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the nucleic acid or protein in the required amount in an appropriate carrier with one or a combination of the ingredients enumerated above, as required, followed by filtered sterilization. Generally, dispersions are prepared by incorporating the active agent into a sterile vehicle, which contains a basic dispersion medium and the required other ingredients from those enumerated above. In the case of sterile powders for the preparation of sterile injectable solutions, the common methods of preparation are vacuum drying and/or freeze-drying, which yields a powder of the active ingredient plus any additional desired ingredient from a previously sterile-filtered solution thereof.

In some embodiments, the nucleic acids of the disclosure can also be administered by transfection or infection using methods known in the art, including but not limited to the methods described in McCaffrey et al., Nature (2002) 418:6893; Xia et al., Nature Biotechnol. (2002) 20:1006-10; and Putnam, Am. J. Health Syst. Pharm. (1996) 53:151-60, erratum at Am. J. Health Syst. Pharm. (1996) 53:325. This includes ASOs, shRNAs, siRNAs, and vectors that encode ASOs, shRNAs, and/or siRNAs such as plasmids, viral vectors, minicircles, and the like. The protein binding agents and bispecific binding agents of the disclosure can also be administered in the form of a nucleic acid that encodes the agent, such as plasmids, viral vectors, minicircles, mRNA and the like, which may comprise modified bases and linkages to adjust the duration of expression to the desired period.

Due to the sensitivity of the CNS, and the low volume of cerebrospinal fluid, the components, characteristics, and volumes used to manufacture formulations for intrathecal administration are tightly constrained. Typically, intrathecal formulations are water-based products with a pH of about 5-7, osmolarity of about 300 mOsM, and minimal adjuvants (such as surfactants, preservatives, antioxidants, and antimicrobial compounds). Surfactants and detergents such as Tween® and polyethylene glycol, and common solubilizing agents such as dimethylformamide and dimethylsulfoxide, cannot be routinely considered safe, and must be evaluated on a case-by-case basis. (T. L. Yaksh et al., Cur Neuropharmacol (2017) 15(2):232-59.) However, antisense oligonucleotides such as nusinersen are now administered intrathecally, and provide guidance for other ASOs (see, e.g., E. E. Neil et al., J Pediatr Pharmacol Ther (2019) 24(3):194-203; C. F. Bennett et al., Ann Rev Neurosci (2019) 42:385-406). In some embodiments, the carrier suitable for intrathecal administration consists essentially of purified water. In some embodiments, the purified water is water for injection. In some embodiments, the carrier further comprises NaCl. In some embodiments, the carrier consists essentially of phosphate buffered saline. In some embodiments, the carrier does not comprise an antimicrobial agent. In some embodiments, the carrier does not comprise a preservative.

ASOs, including siRNA and shRNA, can also be administered to the CNS intranasally, for example using a cationic nanoemulsion (J. H. Azambuja et al., Mol Neurobiol (2019) doi: 10.1007/s12035-019-01730-6), or mesenchymal stem cell-derived exosomes (S. Guo et al., ACS Nano (2019) doi: 10.1021/acsnano.9b01892). Other formulations, such as rabies viral glycoprotein (RVG) exosome formulations, can be administered systemically and target the CNS (M. Izco et al., Mol Ther (2019) doi.org/10.1016/j.ymthe.2019.08.010; C. Liu et al., Theranostics (2019) 9(4):1015-28; J Yang et al., Mol Ther Nucleic Acids (2017) 7:278-87; J. M. Cooper et al., Mov Disord (2014) 29(12):1476-85). See also F. Erdö et al., Brain Res Bull (2018) 143:155-70; R. L. Juliano, Nuc Acids Res (2016) 44(14):6518-48; and P. Boisguérin et al., Adv Drug Deliv Rev (2015) 87:52-67 for additional formulations and methods of administration.

In another aspect, provided herein are formulations for use in connection with CD19-targeted therapy. In some embodiments, the formulation comprises a pharmaceutically acceptable carrier and an effective amount of: (a) a binding agent of the disclosure; (b) an expression modulator of the disclosure; and/or (c) a bispecific binding agent of the disclosure. In some embodiments, the formulation effective amount is sufficient to reduce the number of neurovascular pericytes and/or vSMCs that are killed or incapacitated by the CD19-targeted therapy in vitro by about 10%, about 20%, about 30%, about 40%, about 50%, about 60%, about 70%, about 80%, about 90%, or about 100%. In some embodiments, the agent reduces the number of neurovascular pericytes and/or vSMCs that are killed or incapacitated by the CD19-targeted therapy in an in vivo animal model by about 10%, about 20%, about 30%, about 40%, about 50%, about 60%, about 70%, about 80%, about 90%, or about 100%. In some embodiments, the agent reduces the disruption of the BBB by the CD19-targeted therapy by about 10%, about 20%, about 30%, about 40%, about 50%, about 60%, about 70%, about 80%, about 90%, or about 100%, as measured in an in vivo animal model using exclusion of a marker as a measure of BBB permeability. In some embodiments, the BBB disruption is measured using Evans Blue dye exclusion. In some embodiments, the formulation is suitable for intrathecal or intracerebral administration. In some embodiments, the formulation is suitable for intranasal administration.

E. Kits

In another aspect, provided herein are kits for treating a subject having a CD19⁺ B-cell hyperproliferative disorder, comprising a CD19-targeted therapy, a protective agent, and printed instructions for using the CD19-targeted therapy and/or the protective agent. In some embodiments, the kit comprises a protective agent that: (a) binds to CD19 and reduces binding of the CD19-targeted therapy to CD19⁺ neurovascular pericytes and/or CD19⁺ vSMCs; (b) down-regulates expression of CD19 by neurovascular pericytes and/or vSMCs; or (c) comprises a bispecific binding agent that binds to a non-CD19 pericyte and/or vSMC surface protein and an immune checkpoint surface protein, wherein binding to the immune checkpoint protein reduces the CD19-targeted therapy neurotoxicity. In some embodiments, the protective agent is provided in a formulation. In some embodiments, the formulation is suitable for intrathecal administration.

In another aspect, provided herein is the use for treating a subject for a CD19⁺ B-cell hyperproliferative disorder, of a therapy comprising a CD19-targeted therapy, and a protective agent. In some embodiments, the use is wherein the protective agent (a) binds to CD19 and reduces binding of the CD19-targeted therapy to CD19⁺ neurovascular pericytes and/or CD19³⁰ vSMCs; (b) down-regulates expression of CD19 by neurovascular pericytes and/or vSMCs; or (c) comprises a bispecific binding agent that binds to a non-CD19 pericyte and/or vSMC surface protein and an immune checkpoint surface protein, wherein binding to the immune checkpoint protein reduces the CD19-targeted therapy neurotoxicity. In some embodiment, the use is wherein the protective agent is provided in a formulation. In some embodiments, the use is wherein the formulation is suitable for intrathecal administration.

In another aspect, provided herein is the use of a therapy comprising a CD19-targeted therapy, and a protective agent for the manufacture of a medicament for the treatment of a subject for a CD19⁺ B-cell hyperproliferative disorder. In some embodiments, the use is wherein the protective agent (a) binds to CD19 and reduces binding of the CD19-targeted therapy to CD19⁺ neurovascular pericytes and/or CD19⁺ vSMCs; (b) down-regulates expression of CD19 by neurovascular pericytes and/or vSMCs; or (c) comprises a bispecific binding agent that binds to a non-CD19 pericyte and/or vSMC surface protein and an immune checkpoint surface protein, wherein binding to the immune checkpoint protein reduces the CD19-targeted therapy neurotoxicity. In some embodiments, the use is wherein the protective agent is provided in a formulation. In some embodiments, the use is wherein the formulation is suitable for intrathecal administration.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited herein.

Additional embodiments are disclosed in further detail in the following examples, which are provided by way of illustration and are not in any way intended to limit the scope of this disclosure or the claims.

### Example 1

### Expression of CD19 by Neurovascular Pericytes and vSMCs

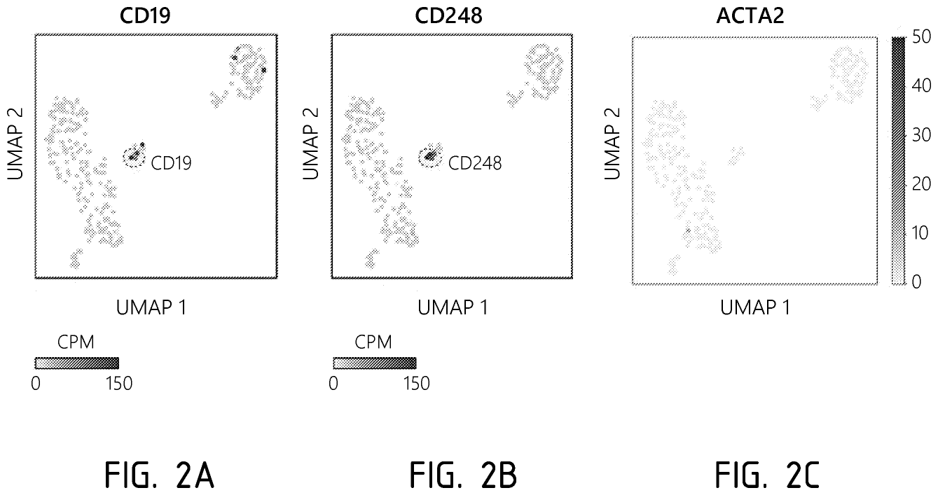

Single-cell RNA-sequencing data from 2,364 human prefrontal cortex cells (S. Zhong et al., Nature (2018) 555:524-28) was analyzed. Cells were clustered and broad populations identified, focusing subsequent analyses on non-neuronal, non-erythroid cells. These were further segregated into astrocyte, lymphocyte, microglial, oligodendrocyte precursor (OPC), endothelial, and pericyte populations (FIG. 1). The populations were identified based on the expression of canonical marker genes: pericytes specifically expressed expected marker genes, such as PDGFRB, FOXF2, RGS5, and CD248, while endothelial cells expressed a distinct set of markers, such as CD34 and PECAM1 (CD31). Surprisingly, this analysis revealed a small population of cells (˜1.5% of non-neuronal cells; ˜0.2% including neuronal cells) that expressed CD19 (FIG. 2A) and co-expressed the pericyte marker CD248 (FIG. 2B). Importantly, this population was negative for the B cell marker CD79A, reducing the possibility of artifactual CD19 expression due to B cell-pericyte doublets. Moreover, this population was negative for the vSMC marker ACTA2 (FIG. 2C). A separate population of lymphocytes that expressed the marker genes CD45 (PTPRC), CD3, CD7, and IL2RG (CD132) was also observed, representing a population of T cells. Notably, the expression of lymphocyte markers and pericyte markers were mutually exclusive in the observed clusters, and the expression of CD19 was specific to the pericyte cluster.

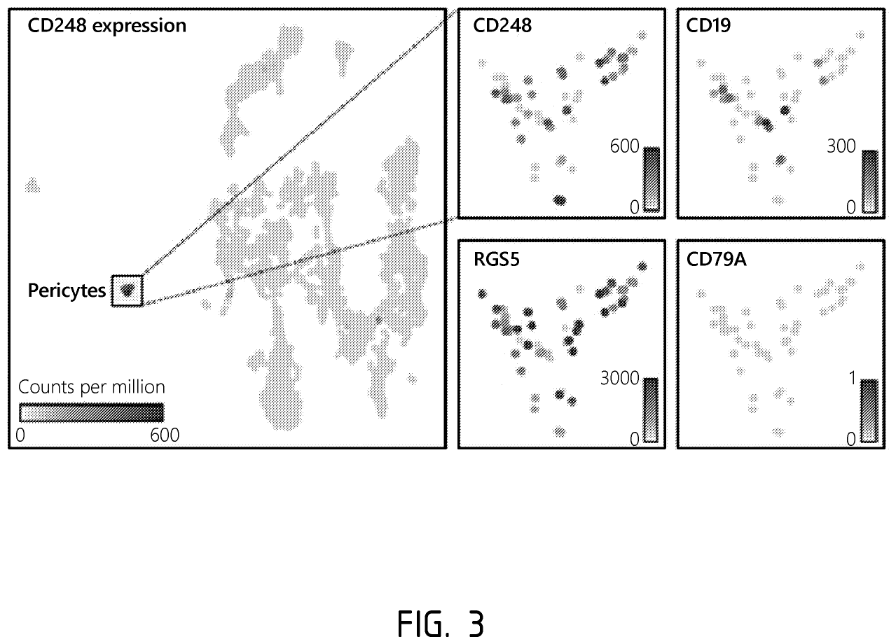

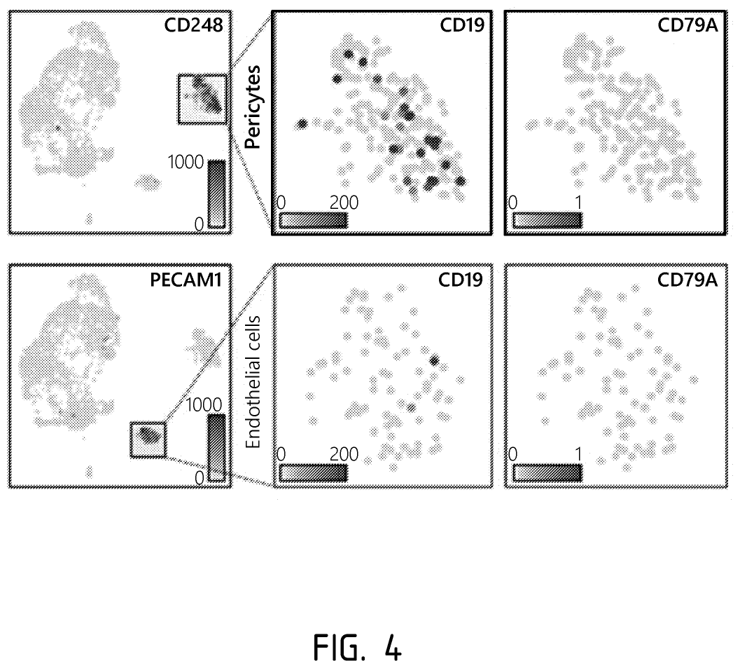

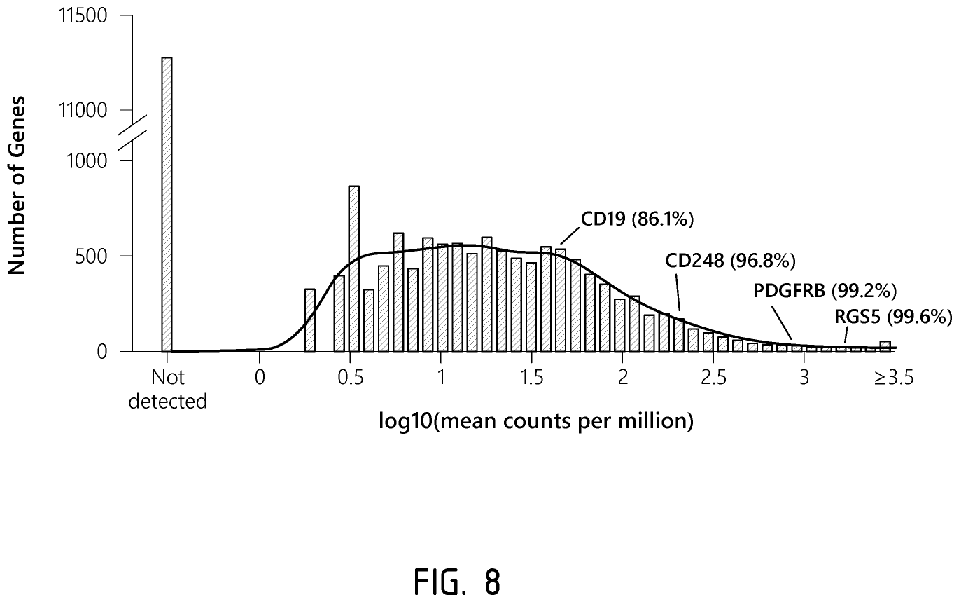

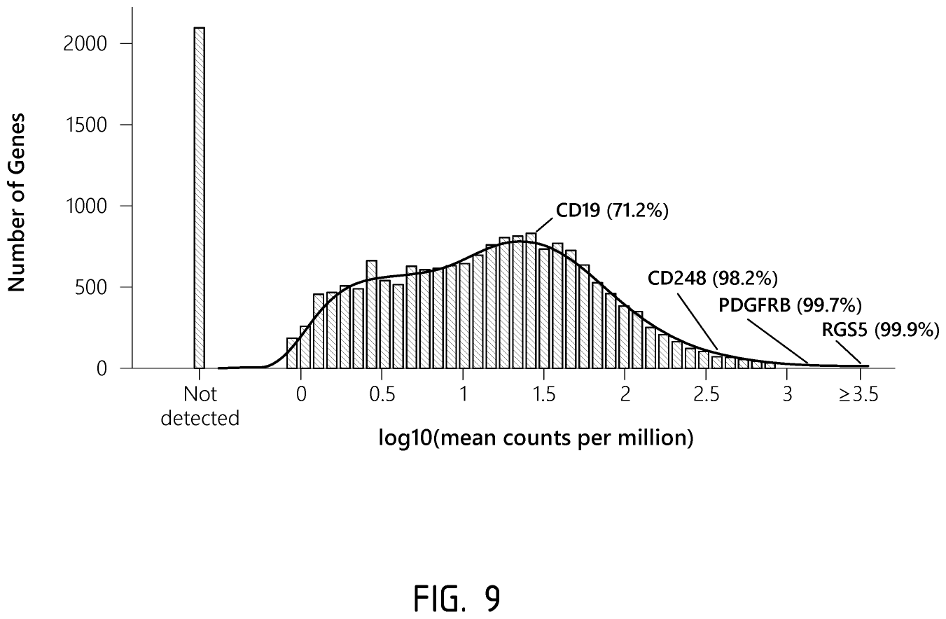

The observation of CD19-expressing pericytes was reproduced in additional independent single-cell RNA-sequencing datasets from the human brain. In a dataset from human forebrain (G. La Manno et al., Nature (2018) 560:494-98), a pericyte population (48/7906 cells) positive for the markers CD248 and RGS5 also displayed CD19 expression (12/48 cells had measurable CD19) and the absence of the B-cell marker CD79A (FIG. 3). In a third dataset derived from human ventral forebrain (G. La Manno et al., Cell (2016) 167:566-90.e19), expression of CD19 was again observed in a population of CD248-positive pericytes (195 pericytes/1977 cells, of which 25/195 had measurable CD19; FIG. 4). As with other datasets, no RNA-sequencing counts for CD79A were identified in any pericytes. Although spurious non-pericyte CD19-positive cells were observable, these were likely the result of cell-cell doublets, such as mural-endothelial cell doublets observed due to the adhesion of mural to endothelial cells in the brain. In these datasets, CD19 ranked in the 86th and 71st percentiles for gene expression, suggesting relatively robust expression (FIGS. 8-9).

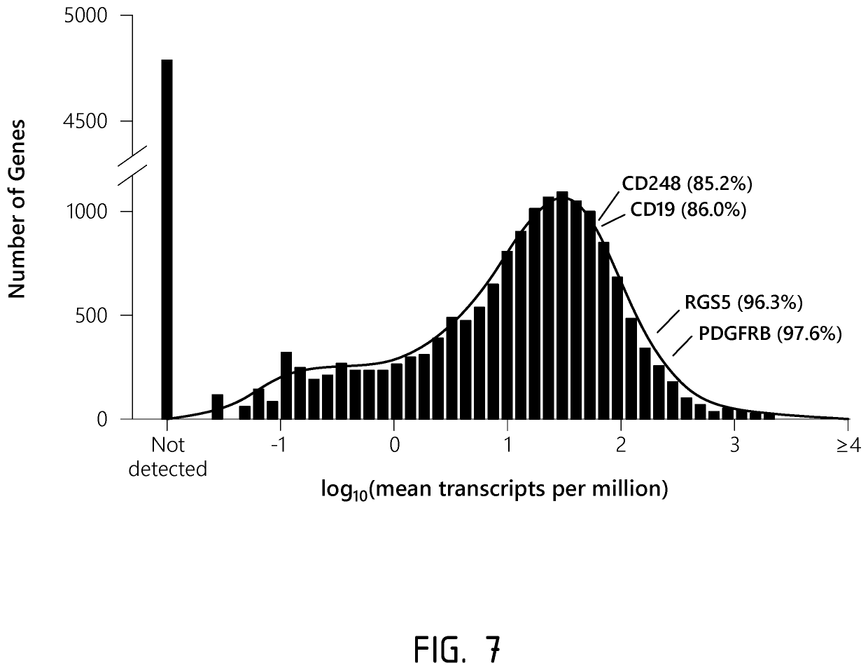

Due to the inherent sparsity of single-cell data, even moderately or highly expressed genes will not be detected in every cell (A. Butler et al., Nature (2010) 468:557-61). Therefore, the level of expression of CD19 in pericytes was compared to known pericyte marker genes relative to the overall gene expression distribution in pericytes. Mean-normalized gene expression values can provide a rough estimate of the relative expression of a given gene in a cell population of interest, despite the inherent sparsity of single-cell data. As expected, pericyte markers such as CD248, RGS5, and PDGFRB ranked in the top percentiles of gene expression in pericytes from the human prefrontal cortex (85th, 96th, and 98th, respectively) (FIG. 7). CD19 ranked in the 86th percentile for gene expression, suggesting relatively robust expression. This finding was repeated, with relatively similar respective gene expression percentiles, in data from the human forebrain datasets.

To assess the expression of CD19 protein in human pericytes and vSMCs, immunohistochemistry was performed on several regions of the human brain using a clinically-validated anti-human CD19 antibody (clone BT51E), which recognizes the C-terminus of the CD19 protein, on samples from healthy deceased subjects. CD19 expression on cells present adjacent to the vessel basement membrane walls was found in perivascular areas. Abluminal CD19 expression was observed across multiple brain regions, with particular regions, such as the hippocampus, insula, temporal lobe, frontal lobe, and parietal lobe displaying a comparatively higher, albeit still rare, incidence of CD19-positive cells. In contrast, regions such as the lower medulla, pons, and occipital lobe displayed lower rates of CD19-positive cells. Notably, CD19-positive cells were found along smaller capillaries (<8 μm) as well as larger vessels (>8 μm; majority of cells depicted), suggesting that in addition to pericytes, CD19 was expressed in vSMCs. The abluminal localization along the vasculature of CD19⁺ cells was most consistent with staining of mural cells.

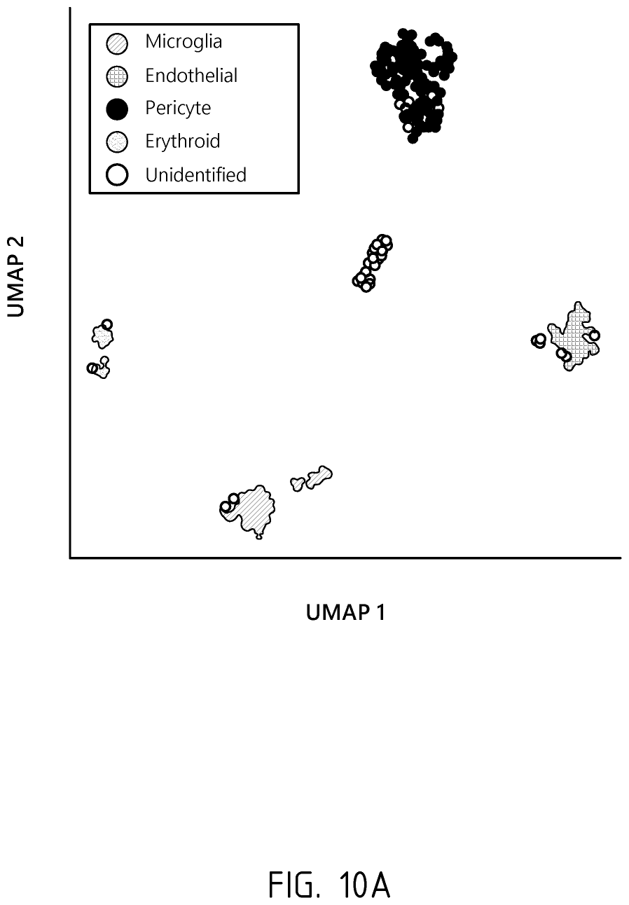

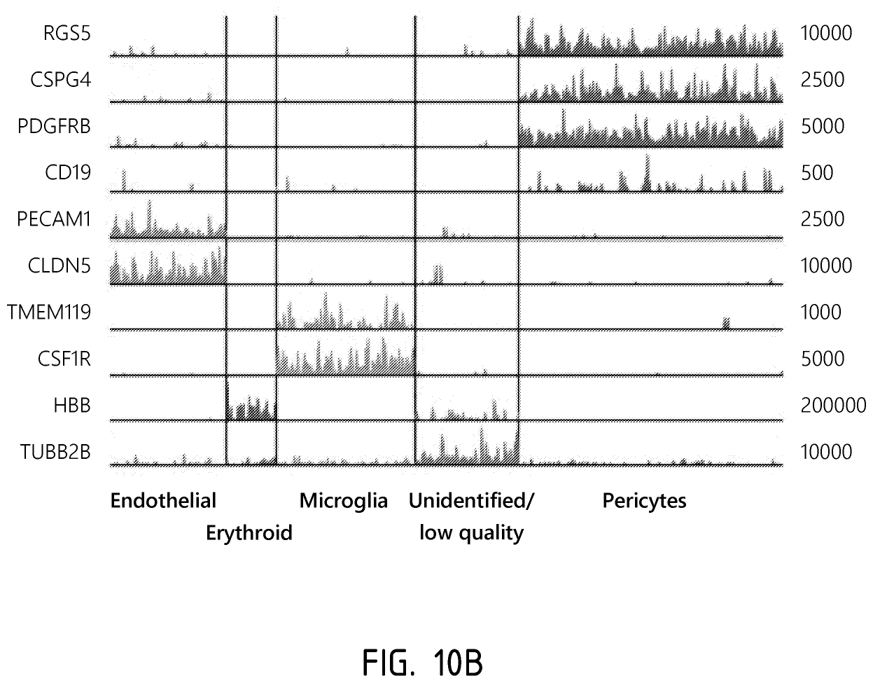

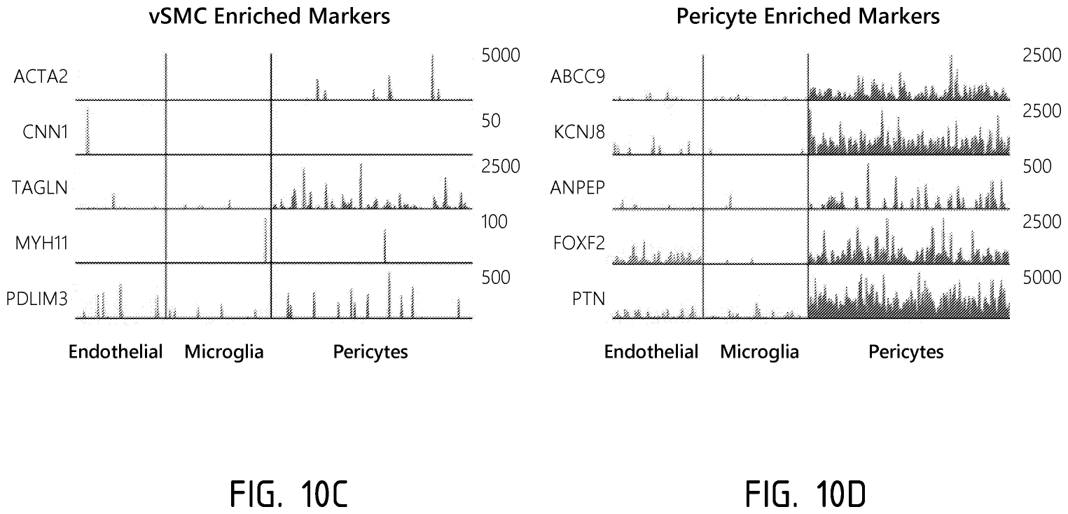

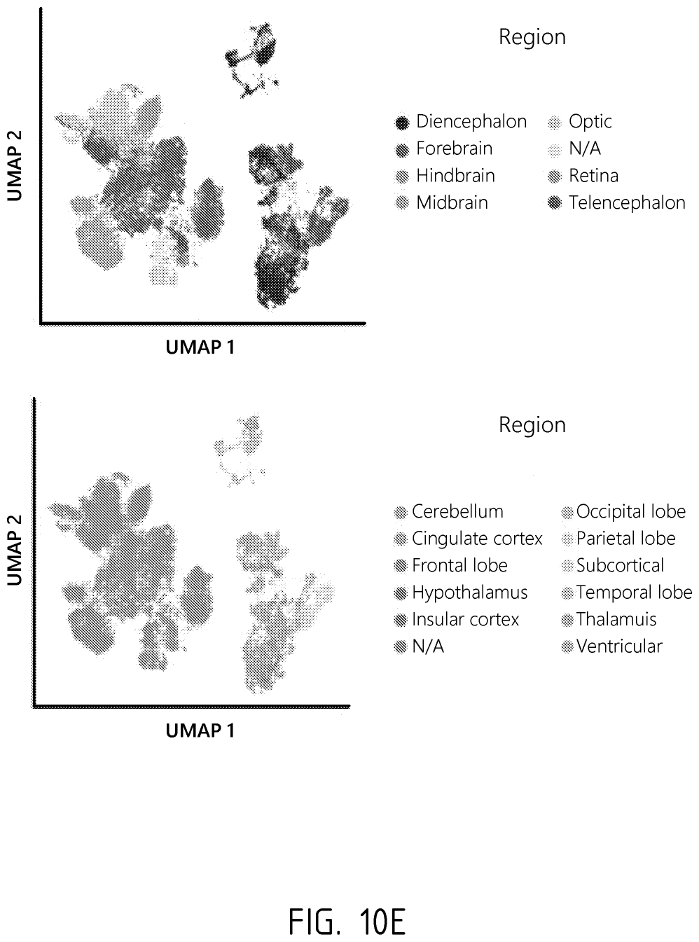

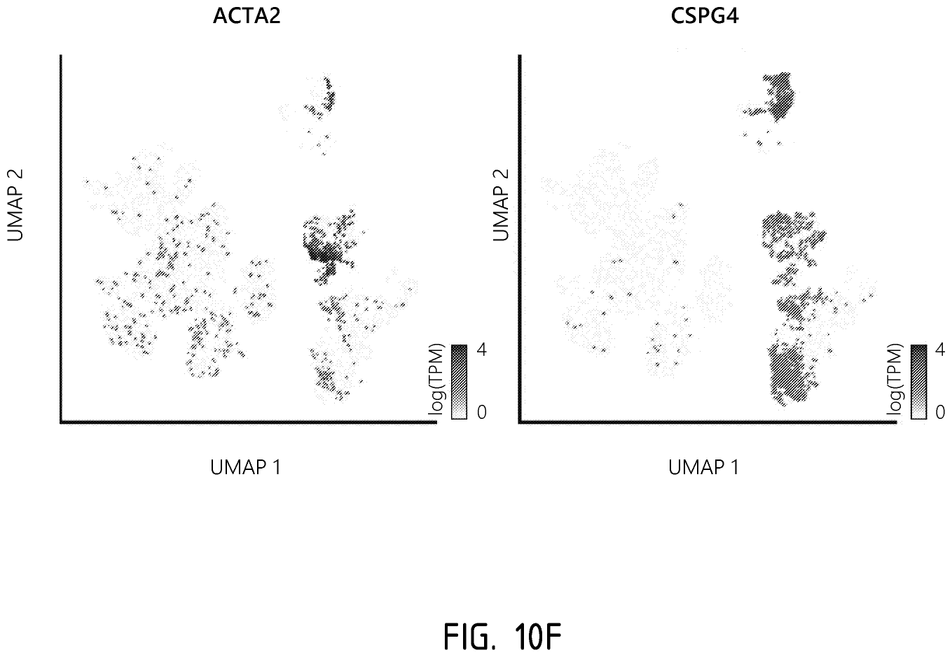

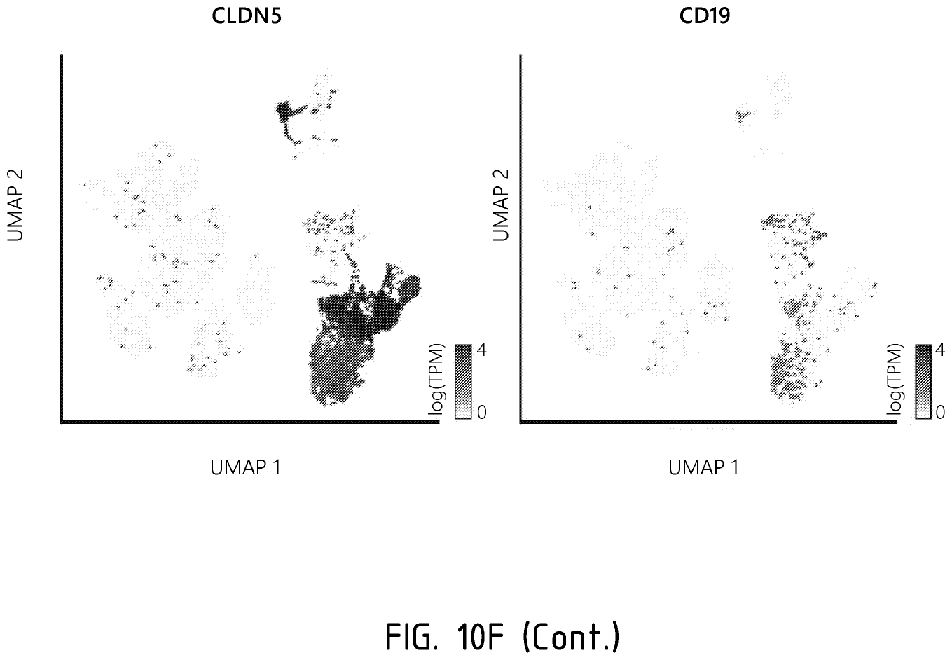

To identify whether the identified CD19⁺ mural cells represented pericytes or vSMCs, the three data sets from Zhong et al., supra (GEO GSE104276) , La Manno et al., 2016, supra (GEO GSE76381), La Manno et al., 2018, supra (PanglaoDB database, Karolinska Institutet) were aggregated and analyzed as a single integrated dataset. A subset of non-neuronal clusters enriched for a mural marker gene CD248. This population expressed both CD19 and CD81. Subsets expressing the endothelial and microglial markers, CD248 and CSF1R, respectively, were also identified. The non-neuronal subset (mural cells, endothelial cells, and microglia) was then re-analyzed and clustered to distinguish between transcriptional differences in cell types of the NVU. These cells showed strong enrichment of pericyte markers, such as ABCC9 and KCNJ8, without enrichment of ACTA2 or other vSMC marker genes, suggesting that they represented bona-fide pericytes (FIGS. 10A-10D).

A detailed analysis of neurovascular cells and related progenitors was performed using the BRAIN Initiative Cell Census Network (BICCN) dataset. A subset of the non-neuronal-biased progenitors, as well as the pericyte and endothelial cell clusters were subjected for further analysis. The subset of cells were representative of cells from many brain regions (FIG. 10E). A smaller vSMC population in this dataset was identified, showing high expression of ACTA2 and TAGLN, as well as other pericyte markers, such as RGS5. Importantly, CD19 was expressed in the vSMC population as well, suggesting that CD19 expression is not unique to pericytes, but a common feature in human brain mural cells (FIG. 10F), consistent with the staining pattern observed in immunohistochemistry.

### Example 2

### CD19 Isoform Recognized by CAR-T Cells in Brain

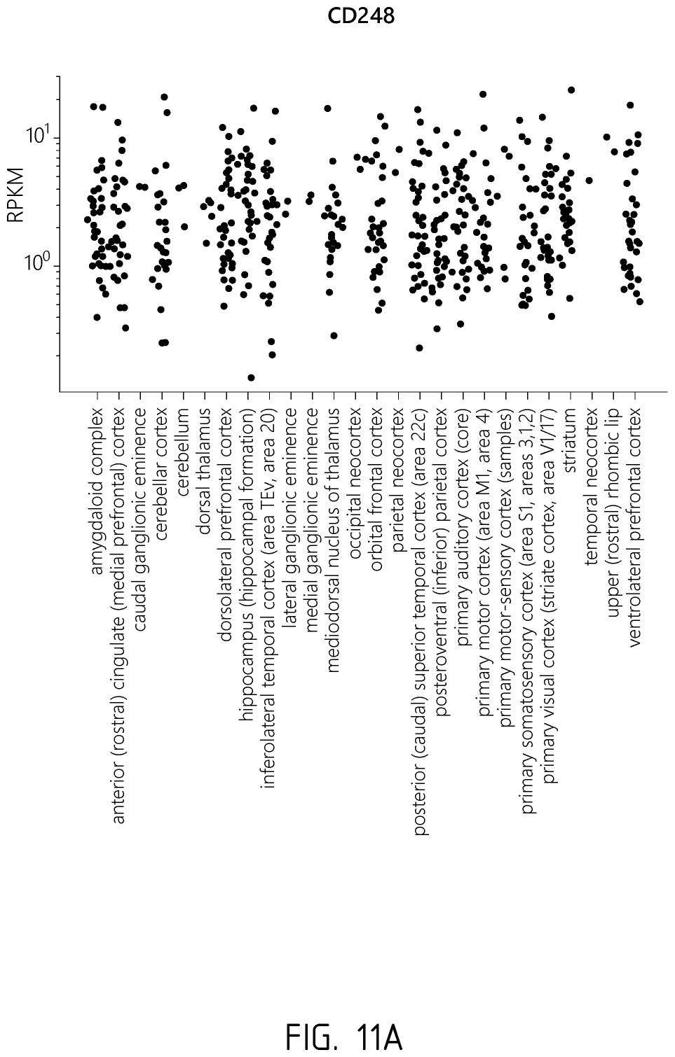

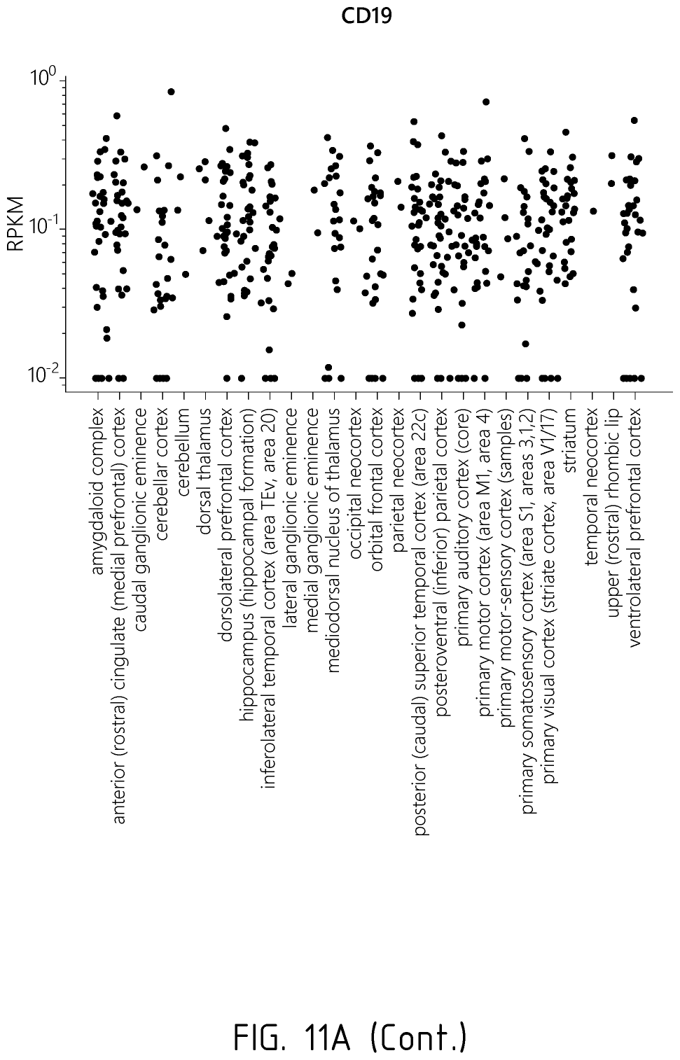

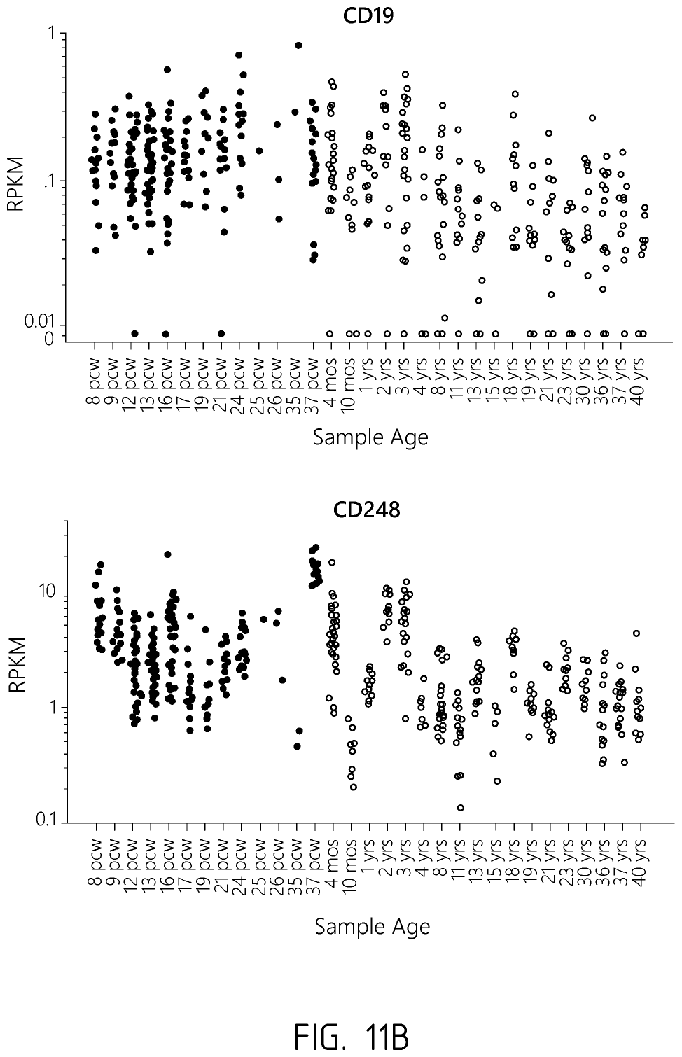

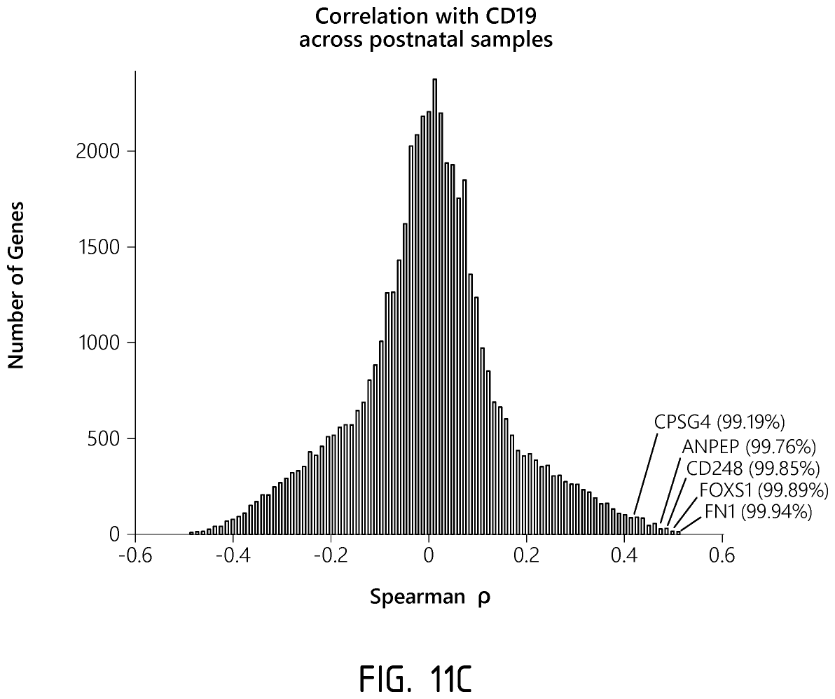

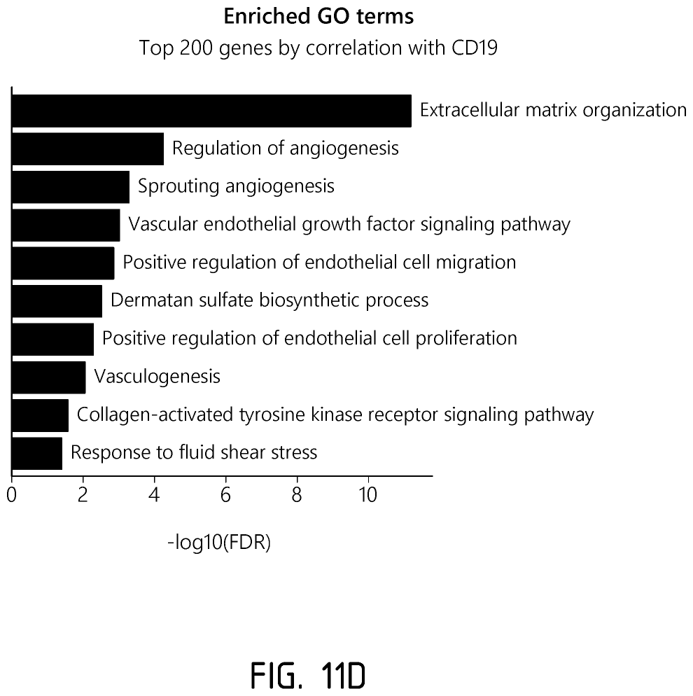

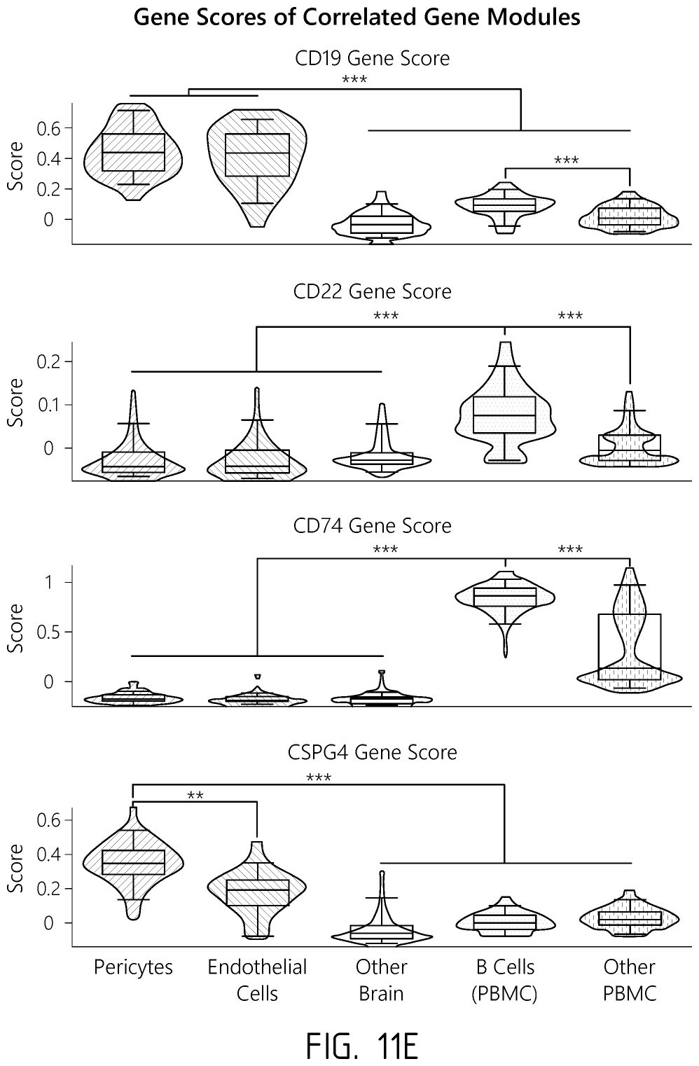

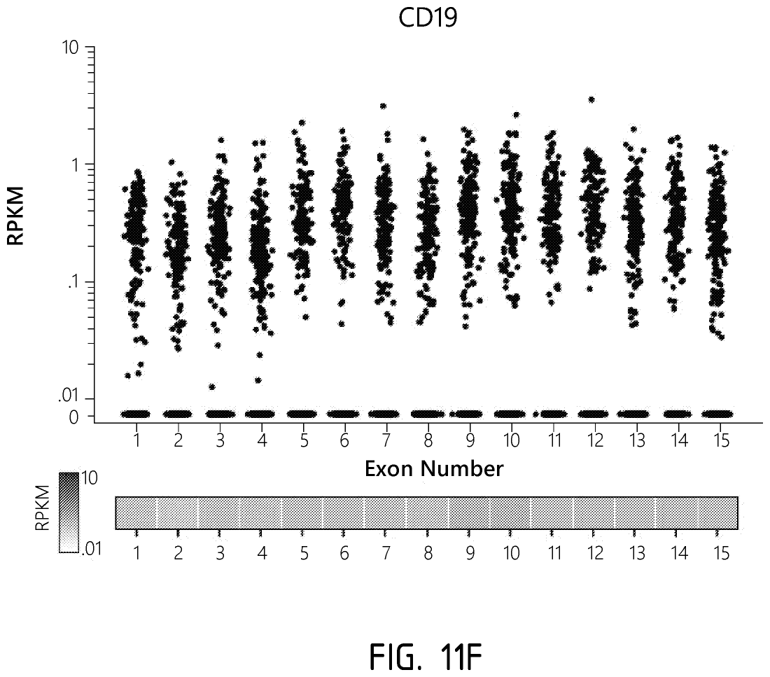

To analyze whether CD19 is recognized by CAR-T cells in the adult human brain, bulk RNA-sequencing data across human age and brain region generated by the Allen Institute Brainspan Project (Miller et al., 2014) was utilized. This data contains more than 500 prenatal and postnatal samples from diverse brain regions (n=237 prenatal; n=287 postnatal). In analyzing samples with varying proportions of mural/vascular cells, the relative expression of mural gens across the samples was a proxy for the underlying proportion of mural cells in the bulk tissue. For example, CD248 and ANPEP, two pericyte markers, were highly correlated in this data. The expression of CD19 is confirmed in both prenatal and postnatal samples at similar levels as well as in different brain regions (FIGS. 11A-11B). A genome-wide correlation analysis across only the postnatal samples was performed to identify genes that are co-expressed with CD19. This analysis showed that CD248, CSPG4, ANPEP, FOXS1, and FN1 were in the top 1% of genes correlated with CD19 (FIG. 11C). Additionally, the top 200 genes most correlated with CD19 were enriched for GO terms associated with the NVU, such as angiogenesis, vasculogenesis, response to fluid shear stress, and multiple extracellular matrix related terms (FIG. 11D). This suggests that the expression of CD19 in the adult brain is primarily the result of mural cell abundance, rather than B cell abundance. The CD19-correlated gene module in adult brain was integrated with RNA sequencing data from the human brain and peripheral blood mononuclear cells (PBMCs). This allowed for the comparison of the gene module score (i.e., the enrichment of genes correlated with CD19 or another target) in brain pericytes, endothelial cells, B cells, in addition to other brain cells and PBMCs. As a control, the gene modules associated with CD22 and CD74, two highly expressed B cell-enriched genes, were enriched in B cells, and the CSPG4 gene module was highly enriched in pericytes. In contrast, the expression of the CD19-correlated gene module is highest in pericytes (FIG. 11E). Whether the CD19 isoform expressed in the adult brain contained the specific CD19 epitope recognized by clinical CAR-T cells and BiTEs was investigated. The FMC63 scFv in clinical use recognizes an epitope encoded by exon 4 of CD19. Additionally, variants skipping exon 2 may also result in a lack of CD19 trafficking to the cell surface, also allowing evasion of FMC63 detection. An analysis of the mean expression of each CD19 exon in the adult human brain showed an even distribution of expression across exons, which clear expression of the key exons 2 and 4 (FIG. 11F).

To complement these observations, brains were extracted from healthy C57B1/6J mice, and stromal cells were isolated from the brain using the method described by A. Boroujerdi et al., Meth Mol Biol (2014) 1135:383-92. Analysis by flow cytometry of live single cells demonstrated the presence of CD19⁺ cells within the pan-CD45 negative fraction, in addition to CD31⁺ endothelial cells. A population of CD45⁺ B cells was also identifiable on the basis of CD19 and B220 expression. The CD19 expression levels among CD19⁺, CD45-cells was comparable to that of CD45⁺, CD19⁺ B-cells, displaying clear separation from CD19 levels in endothelial cells as well as the overall CD45 negative stromal fraction. In contrast, B220 expression was found only in B cells, and not in CD19k, CD45-cells, as expected. Together, these results strongly suggest that CD19-positive cells are present in the brain and appear both transcriptionally and histologically as pericytes and/or vSMCs.

### Example 3

### Human Brain Mural-Specific Expression of CD19

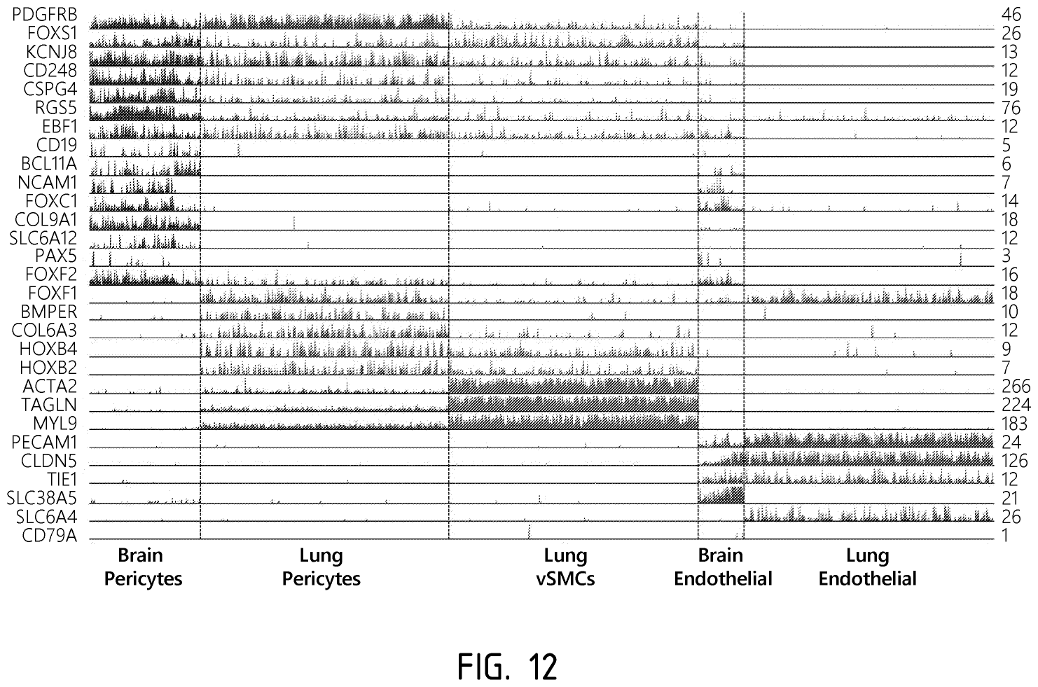

Since mural cells are present in multiple organs, a comparative analysis of brain pericytes with pericytes and vSMCs from the lung, a highly vascularized tissue with high numbers of pericytes and endothelial cells, was performed. Although all mural cell populations showed shared expression of a core transcriptional identity, such as PDGFRB, RGS5, FOXS1, and KCNJ8, numerous transcriptional differences between brain and lung pericytes were identified (FIG. 12). Notably, CD19 was specifically expressed in brain, but not lung mural cells. Only 6/2724 lung mural cells had any detectable counts for CD19, showing no apparent enrichment over nonspecific counts found in all non-B cell clusters. Whether genes predicted to localize to the membrane or cell surface were differentially expressed was investigated in the NVU relative to lung pericytes as well as B cells. Also, whether genes predicted to localize to the membrane or cell surface of mural cells could be used to improve the safety of CD19-directed CAR-T. The most highly differentially expressed genes were identified among those annotated as being secreted or located on the cell surface, in brain versus lung pericytes, as well as B cells versus brain pericytes and endothelial cells. These could improve the specificity of B cell-directed CAR-T cells by requiring dual recognition of two antigens. This analysis revealed additional genes, such as CD74, HLA-DRA, and LTP, which are both highly expressed and highly enriched on B cells relative to brain pericytes. An alternative approach to improving CAR-T cell specificity is to use “NOT” gates, where inhibitory CAR constructs recognize non-specific target proteins and prevent T cell activation. We identified numerous genes that are enriched in pericytes over B cells, such as BGN, FN1, and SEMA5A (FIG. 12).

### Example 4

### BBB Disruption

As CD19-positive non-B cells were also present in the mouse brain, mice lacking a B-cell population were tested to determine whether BBB disruption was observed, in order to control against any BBB disruption resulting from cytokine release syndrome-related symptoms. CD28-based or 4-1BB-based CAR-T cells specific for either murine CD19 (1D3 scFv, mCD1928z and mCD19BBz) or human CD19 (FMC63 scFv, hCD19BBz) were generated (M. C. Milone et al., Mol Ther (2009) 17:1453-64). The mCD19BBz and mCD1928z CARs were constructed by ligating mCD19 scFv (1D3) into the CAR backbone sequences of pTRPE-BBz and pTRPE-28z. The hCD19BBz cells represent a negative control experimental condition, as no CD19-specific targeting would be expected in recipient mice due to the absence of strong sequence homology at the FMC63 epitope targeted by the hCD19BBz cells (D. Sommermeyer et al., Leukemia (2017) 31:2191-99). Human CAR-T cells were produced from normal donor T cells provided by the University of Pennsylvania Human Immunology Core. Cells were transduced with lentiviral vectors encoding anti-human (h) or murine (m) CD19 scFv fused to CAR backbones containing either human 4-1BB or human CD28 and CD3zeta (CD3ζ, CD247) signaling domains, as described (M. C. Milone et al., supra) and were expanded ex vivo for 11 days. Two cell expansions were produced from 2 different healthy human donors. The transduction efficiencies ranged from 20 to 40%.

CAR expression on the cell surface was confirmed, and CAR-T cell functionality tested in vitro using flow cytometric-based cytotoxicity assays with the human CD19⁺ B-ALL cell line Nalm6 and the murine CD19⁺ B-ALL cell line A20 as targets. Specific lysis of Nalm6 cells, but not A20 cells, by anti-human CD19 CAR-T cells with increasing effector-to-target ratios was observed. Conversely, anti-murine CD19 CAR-T cells lysed A20 at a much higher rate than Nalm6 cells. The species specificity of both anti-murine and anti-human CD19 CAR-T cells also confirmed based on IFN-γ secretion.

To address whether BBB disruption is caused by on-target cytotoxicity of a cell type other than B cells, and independent of a CRS-related effect from B cell targeting, immunodeficient, non-tumor bearing NSG mice were treated with PBS, human CAR-T cells containing either CD28-based or 4-1BB-based constructs specific for murine CD19, or human 4-1BB-based CAR-T cells targeting human CD19. Seven days post CAR-T cell infusion, mice were infused intravenously with Evans Blue dye (EBD), which allows quantitative measurement of extravasation and can be used to analyze BBB permeability (see, e.g., V. Braniste et al., Sci Transl Med (2014) 6(263):263ra158). EBD binds to albumin, which remains in the bloodstream in normal physiologic conditions. When the BBB is disrupted, small proteins such as albumin can cross. Mice receiving no CAR-T infusion were treated with mannitol and EBD simultaneously as a positive control of increased BBB permeability.

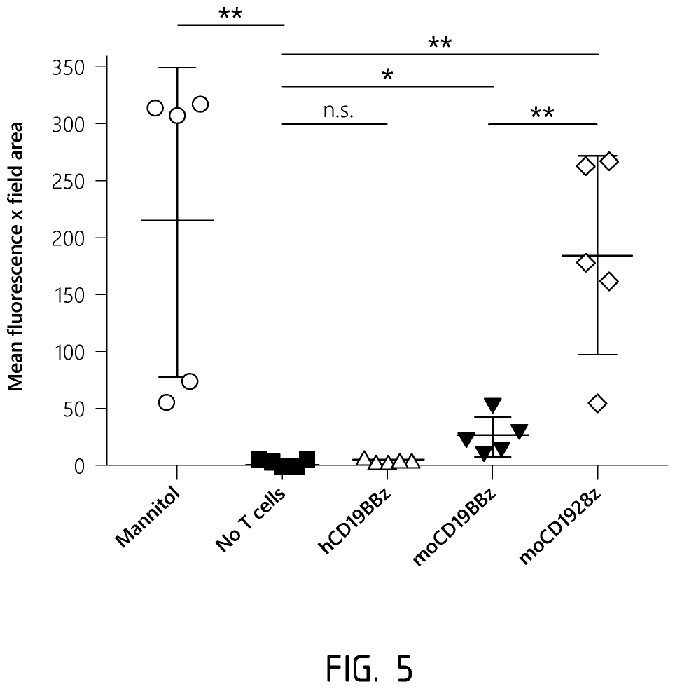

Thirty minutes after EBD injection, mice were euthanized and brains were harvested, formalin-fixed, and paraffin-embedded. Deparaffinized cross sections of brain were stained with DAPI and imaged using confocal microscopy for EBD fluorescence. Mice treated with mannitol displayed EBD fluorescence indicative of BBB extravasation, in contrast to mice receiving no treatment and mice receiving anti-human CD19 CAR-T cells (hCD19BBz). Mice treated with anti-murine mCD19BBz cells, and to a higher extent, anti-murine mCD1928z cells, displayed BBB extravasation indicative of disruption of the BBB independent of any B cell-killing-related effects. These results were quantified, displaying a significant enrichment of EBD fluorescence in mCD19BBz and mCD1928z conditions (FIG. 5).

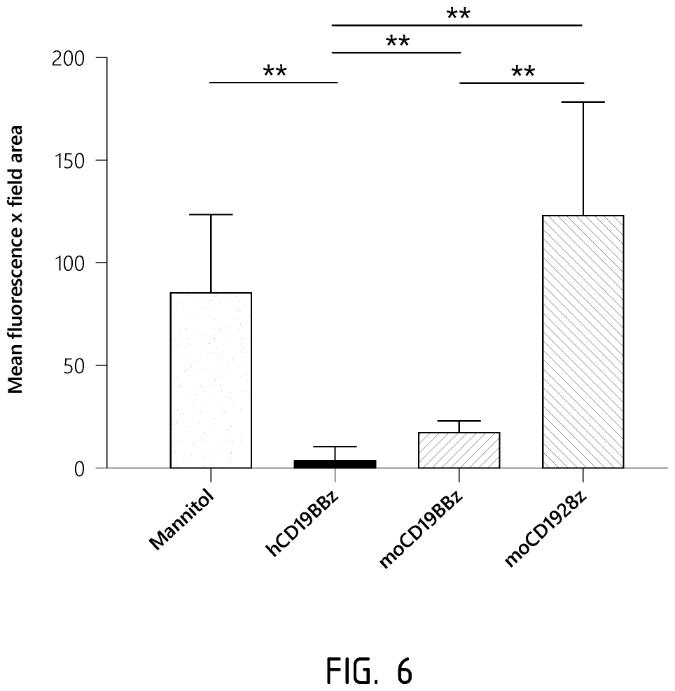

This animal study was repeated using a syngeneic, immunocompetent C57B¹/₆J model with cyclophosphamide as a lymphodepleting preconditioning regimen. In this model, mice were treated with murine T cells expressing murine versions of the CARs evaluated in the NSG model (hCD19BBz, mCD19BBz, or mCD1928z) and analyzed as above. The syngeneic study recapitulated the pattern of BBB permeability observed in the NSG model, showing that the presence of murine CD19⁺ B cells in the syngeneic model did not affect the specific disruption of the BBB observed only in the murine-targeting conditions (FIG. 6).

The BBB integrity after CAR-T cell treatment was also measured using high-definition imaging with 9.4 tesla magnetic resonance imaging (MRI). Immunodeficient, non-tumor bearing NSG mice were treated with PBS, mannitol, hCD19BBz, mCD19BBz, or mCD1928z conditions, as before. Four days post infusion, brain MRI analysis confirmed an increase in gadolinium uptake in the brain parenchyma in the mCD19BBz and mCD1928z conditions as well as the mannitol control. As before, the hCD19BBz condition did not display BBB disruption, and the CD28-based CAR-T cells displayed increased BBB disruption relative to the 4-1BB-based CAR-T cells.

### Example 5

### Protective Agent Design

1. Antigen Identification

Gene expression values of proteins reported to be on the cell surface or secreted in pericytes, vSMCs, endothelial cells, and B cells, were compared in order to identify putative markers that might be used in an inhibitory CAR setting alongside an activating CD19 CAR (G. X. Y. Zheng, Nature Com (2017) 8:14049). As the complete and exact repertoire of cell surface proteins found on a given cell type is inherently difficult to know comprehensively, genes identified on the cell surface by mass spectrometry were included for analysis (D. Bausch-Fluck et al., PLoS ONE (2015) 10:e0121314).

Processed sequencing data (gene counts per cell) were downloaded as follows: Zhong et al., supra (GEO GSE104276), La Manno et al., 2016, supra (GEO GSE76381), La Manno et al., 2018, supra (PanglaoDB database, Karolinska Institutet). Samples were processed using Seurat version 2.3.4 (A. Butler et al., Nat Biotechnol (2018) 36:411-20) and Scanpy version 1.3.1 (F. A. Wolf et al., Genome Biol (2018) 19:15). Cells with fewer than 500 detected genes or UMI counts were excluded, and cell counts were normalized per cell. The 1500-2500 most variable genes were used for clustering based on the variance to mean ratio. As the datasets include both post-mitotic and actively cycling cells, the cell cycle status was computed using the CellCycle-Scoring function and subsequently regressed out using the ScaleData function in Seurat.

Principle component analysis was performed using the genes identified as highly variable for each dataset, and the top ˜25-50 principle components were used for subsequent dimensionality reduction using the UMAP algorithm. Clusters were called using the FindClusters function in Seurat, and marker genes for each cluster were identified using the FindMarkers function. Clusters were subsequently manually annotated by comparing highly enriched genes to known cell-type markers. For the analysis of Zhong et al. 2018 data, neuronal precursor cells, erythroid cells, and neuronal cells were identified and excluded, and the remaining cells were subsequently re-clustered. Gene expression data shown in FIG. 7 represents mean expression across all cells with a given cluster label. Percentages and cell counts of a given label (e.g., pericytes and/or vSMCs) represent the total number of cells in a given cluster, not necessarily only those cells positive for an individual marker, unless otherwise indicated.

Single-cell gene expression data from B cells (G. X. Y. Zhen et al, supra) was obtained from the 10× Genomics website. A database of extracellular proteins based on mass spectrometry data was used for the analysis of putative cell-surface proteins (D. Bausch-Fluck et al., supra). This database included proteins that may be secreted and thus not strongly enriched at the cell surface, but in the interest of not excluding proteins that might be at the cell surface, this more comprehensive database was used. Gene expression was computed as the mean across all single B cells. To account for differences in the expected distribution of mean gene expression due to the inherent sparsity of single-cell RNA expression data, which is biased based on cell capture and library preparation technique, as well as sequencing depth, the distribution of gene expression values were quantile normalized using the normalize.quantiles function in R to facilitate comparisons across cell types. As such, the absolute gene expression values are pseudo arbitrary, in that they have been transformed to be relatively comparable across different cell types from distinct single-cell sequencing techniques.

Automated immunohistochemistry was performed with Ventana Benchmark XT following a clinically-validated protocol for CD19. FFPE tissue sections (4 μm) were deparaffinized and rehydrated. Antigen retrieval was performed using Standard Cell Condition 1 (pH 8.5) for 60 minutes (Ventana Medical Systems). Slides were incubated with anti-human CD19 (1:50, monoclonal, Abnova BT51E) at 37° C. for 30 minutes. The ultraView Universal DAB Detection system (Ventana) was used with 3,3′-diaminobenzidine chromogen.

This analysis revealed an expected enrichment of HLA genes in B cells, as well as the moderately higher expression of CD19 in B cells compared to pericytes, as expected. It also identified genes such as Biglycan (BGN), Fibronectin (FN1), and Semaphorin 5A (SEMA5A) as being highly expressed in pericytes but not in B cells, alongside many other genes enriched in either pericytes or endothelial cells relative to B cells. Lastly, immunostaining for CD19 was performed on tissue from healthy human hippocampus. This showed positive staining for CD19 on cells positioned proximal to the abluminal surface of thin-walled vessels, consistent with the perivascular localization of pericytes.

Based on the differential expression data, BGN, FN1, and SEMASA are selected as the pericyte targets for the construction of bispecific binding agents and safety receptors for CD19-targeted CAR-T. The known pericyte markers, CD248, RGS5, NG2, are also selected for comparison.

2. Immune Cell Targets

The following T cell checkpoint targets are evaluated: CTLA-4, A2AR, VTCN1, BTLA, PD-1, LAG3, 2B4, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, and TIM-3. Agonists of CTLA-4 are prepared as described by B. T. Fife et al., J Clin Invest (2006) 116(8):2252-61 (scFv); S. J. Shieh et al., J Immunol (2009) 183(4):2277-85 (scFv); along with CTLA-4-binding fragments of CD80 and CD86. Agonists of A2AR are prepared by generating antibodies, or (in the case of bispecific agents) can use a small molecule agonist such as CGS-21680 (Tocris) or ZM-241385 (Tocris) (C. Sorrentino et al., Front Immunol (2019) 10:162). Agonists of PD-1 are described by S. Shibiyama et al., U.S. Pat. No. 9,701,749, C. Wood et al., U.S. Pat. No. 6,808,710, and B. M. Carreno et al., U.S. Pat. No. 7,029,674. Agonists of BTLA are described by J. C. Albring et al., J Exp Med (2010) 207(12):2551-59, and J. M. Mataraza et al., U.S. Pat. No. 10,155,813. Agonists of the other proteins are prepared by analogous methods.

3. Pericyte and/or vSMC Targets

Antibody derivatives specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3 are designed as scFv agents. Polynucleotides encoding the a pericyte scFv and an immune cell target scFv are then synthesized, and expressed and administered in a manner analogous to BiTE® agents.

### Example 6

### siRNA

Two different anti-CD19 siRNA molecules are synthesized, V1 (CCGGCTTCAACGT-CTCTCAACAGATCTCGAGATCTGTTGAGAGACGTTGAAGTTTTTG, SEQ ID NO:1) and V2 (CCGGTCAAGACGCTGGAAAGTATTACTCGAGTAATACTTTCCAGCGTCTTGATTTTTTG, SEQ ID NO:2). Non-tumor bearing NSG mice are treated with either V1, V2, or vehicle by intrathecal administration, and allowed to recover for 24 hours.

As in Example 4 above, the mice are then treated with PBS, human CAR-T cells containing either mCD1928z or mCD19BBz specific for murine CD19, or hCD19BBz CAR-T cells targeting human CD19. Seven days post CAR-T cell infusion, mice are infused intravenously with EBD to analyze BBB permeability. Mice receiving no CAR-T infusion are treated with mannitol and EBD simultaneously as a positive control of increased BBB permeability.

Thirty minutes after EBD injection, mice are euthanized and brains are harvested, formalin-fixed, and paraffin-embedded. Deparaffinized cross sections of brain are stained with DAPI, and imaged using confocal microscopy for EBD fluorescence. The results are quantified and the degree of reduction in BBB disruption is determined.

What is claimed is:

1. A method for aiding in the treatment of a B-cell hyperproliferative disorder in a subject, using a CD19-targeted therapy, the method comprising:
(a) administering an effective amount of an agent to the subject, wherein
(i) the agent is a binding agent which binds to CD19 and reduces binding of the CD19-targeted therapy to CD19⁺ neurovascular pericytes and/or CD19⁺ vascular smooth muscle cells (vSMCs); or
(ii) the agent is an expression modulator that down-regulates or eliminates the expression of CD19 by neurovascular pericytes and/or vSMCs; or
(iii) the agent is a bispecific binding agent that comprises a first binding domain that activates an immune checkpoint surface protein, and a second binding domain having affinity for a non-CD19 pericyte surface protein and/or a non-CD19 vSMC surface protein; and
(b) administering a therapeutically effective amount of the CD19-targeted therapy.

2. The method of claim 1, wherein the agent is a binding agent that comprises an antibody or an antibody derivative that binds to CD19.

3. The method of claim 2, wherein the binding agent comprises an antibody derivative.

4. The method of claim 2 or 3, wherein the antibody derivative is a nanobody, duobody, diabody, triabody, minibody, F(ab′)2 fragment, Fab fragment, single chain variable fragment (scFv), or a single domain antibody (sdAb).

5. The method of any one of claims 1 to 4, wherein the binding agent comprises a nanobody or an scFv.

6. The method of claim 1, wherein the agent is an expression modulator that down-regulates or eliminates expression of CD19 by neurovascular pericytes or vSMCs and comprises an antisense oligonucleotide (ASO), an siRNA, or a shRNA.

7. The method of claim 1 or 6, wherein the expression modulator agent comprises a single stranded ASO.

8. The method of claim 1 or 6, wherein the expression modulator agent comprises an siRNA.

9. The method of claim 1 or 6, wherein the expression modulator agent comprises an shRNA.

10. The method of any one of claims 1 to 9, wherein the agent is administered intrathecally.

11. The method of claim 1, wherein the agent is a bispecific binding agent, and the immune checkpoint surface protein comprises PD-1, CTLA-4, ICOS, LAG-3, 2B4, BTLA, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP or TIM-3.

12. The method of claim 11, wherein the first binding domain comprises a CTLA-4-binding domain of CD80 or CD86.

13. The method of claim 11, wherein the first binding domain comprises a CTLA-4 agonist.

14. The method of claim 13, wherein the CTLA-4 agonist comprises an antibody or an antibody derivative.

15. The method of claim 11, wherein the first binding domain comprises a PD-1 binding domain of PD-L1 or PD-L2, or an antibody or an antibody derivative specific for PD-1.

16. The method of claim 15, wherein the first binding domain comprises a PD-1 agonist.

17. The method of claim 11, wherein the first binding domain comprises an A2AR binding agent.

18. The method of claim 17, wherein the A2AR binding agent comprises an antibody or a derivative thereof specific for A2AR.

19. The method of claim 17 or 18, wherein the A2AR binding agent comprises an A2AR agonist.

20. The method of any one of claims 11 to 19, wherein the non-CD19 pericyte surface protein and/or the non-CD19 vSMC surface protein comprise BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

21. The method of any one of claims 11 to 20, wherein the second domain comprises an antibody or a derivative thereof specific for BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

22. The method of any one of claims 1 to 21, wherein the agent is administered at about the time of administering the CD19-targeted therapy.

23. The method of any one of claims 1 to 22, wherein the agent is administered about 15, 14, 13, 12, 11, 10, 9, 8, 7, 6, 5, 4, 3, 2 or 1 days prior to administering the CD19-targeted therapy.

24. The method of any one of claims 1 to 23, wherein the agent is administered about 1, 2, 3, 4, 5, 6, 7, 8, 9, or 10 days after administering the CD19-targeted therapy.

25. The method of any one of claims 1 to 24, wherein the agent is administered multiple times between about 15 days prior and about 10 days after administering the CD19-targeted therapy.

26. The method of any one of claims 1 to 25, wherein the agent is administered after the subject presents signs of neurotoxicity.

27. The method of any one of claims 1 to 26, wherein the agent reduces the number of neurovascular pericytes and/or vSMCs that are killed or incapacitated by the CD19-targeted therapy in vitro by about 10%, about 20%, about 30%, about 40%, about 50%, about 60%, about 70%, about 80%, about 90%, or about 100%.

28. The method of any one of claims 1 to 26, wherein the agent reduces the number of neurovascular pericytes and/or vSMCs that are killed or incapacitated by the CD19-targeted therapy in an in vivo animal model by about 10%, about 20%, about 30%, about 40%, about 50%, about 60%, about 70%, about 80%, about 90%, or about 100%.

29. The method of any one of claims 1 to 28, wherein the agent reduces the disruption of the blood-brain barrier (BBB) by the CD19-targeted therapy by about 10%, about 20%, about 30%, about 40%, about 50%, about 60%, about 70%, about 80%, about 90%, or about 100%, as measured in an in vivo animal model using exclusion of a marker as a measure of BBB permeability.

30. The method of claim 29, wherein the BBB disruption is measured using Evans Blue dye exclusion.

31. The method of any one of claims 1 to 30, wherein the agent is administered as a formulation that comprises:
(a) an agent which
(i) binds to CD19 and reduces binding of the CD19-targeted therapy to CD 19+ neurovascular pericytes and/or CD19⁺ vSMCs; or
(ii) down-regulates the expression of CD19 by pericytes or vSMCs; and
(b) a carrier suitable for intrathecal administration.

32. The method of any one of claims 1 to 31, wherein the CD19-targeted therapy comprises an anti-CD19 chimeric antigen receptor T cell (CAR-T) or a bispecific T cell engager specific for CD19.

33. A formulation for use in connection with a CD19-targeted therapy, the formulation comprising:
an agent which
(i) binds to CD19 and reduces binding of the CD19-targeted therapy to CD19⁺ neurovascular pericytes and/or CD19⁺ vSMCs; or
(ii) down-regulates the expression of CD19 by pericytes and/or vSMCs; and a carrier suitable for intrathecal administration.

34. The formulation of claim 33, wherein the formulation does not contain an antimicrobial agent or a preservative.

35. A system for treating a B-cell hyperproliferative disorder in a subject, while preserving the subject's BBB, the system comprising:
(a) a CD19-targeted therapy, and
(b) an agent selected from:
(i) a binding agent that binds to CD19 and reduces binding of the CD19-targeted therapy to CD19⁺ neurovascular pericytes and/or CD19⁺ vSMCs; or
(ii) an expression modulator which down-regulates the expression of CD19 by neurovascular pericytes and/or vSMCs; and
(iii) a bispecific binding agent that comprises a first binding domain that activates an immune checkpoint surface protein, and a second binding domain having affinity for a non-CD19 pericyte surface protein and/or a non-CD19 vSMC surface protein.

36. The system of claim 35, wherein the CD19-targeted therapy comprises an anti-CD19 chimeric antigen receptor T cell (CAR-T) or a bispecific T cell engager specific for CD3 and CD19.

37. The system of claim 35 or 36, wherein the agent comprises an antibody or an antibody derivative that binds to CD19.

38. The system of any one of claims 35 to 37, wherein the antibody derivative is a nanobody, duobody, diabody, triabody, minibody, F(ab′)₂ fragment, Fab fragment, single chain variable fragment (scFv), or a single domain antibody (sdAb).

39. The system of any one of claims 35 to 38, wherein the agent comprises a nanobody or an scFv.

40. The system of claim 35 or 36, wherein the agent is an expression modulator and comprises an antisense oligonucleotide (ASO), an siRNA, or a shRNA.

41. The system of any one of claim 35, 36, or 40, wherein the agent comprises an siRNA.

42. The system of any one of claim 35-36 or 40-41, wherein the agent comprises an shRNA.

43. The system of any one of claim 35-36 or 40-42, wherein the agent comprises an ASO.

44. The system of any one of claims 35 to 43, wherein the agent is provided in a formulation suitable for intrathecal administration.

45. The system of claim 35, wherein the agent is a bispecific binding agent and the immune checkpoint surface protein comprises PD-1, CTLA-4, ICOS, LAG-3, 2B4, BTLA, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, or TIM-3.

46. The system of claim 45, wherein the first binding domain comprises a CTLA-4-binding domain of CD80 or CD86.

47. The system of claim 45 or 46, wherein the first binding domain comprises a CTLA-4 agonist.

48. The system of claim 47, wherein the CTLA-4 agonist comprises an antibody or an antibody derivative.

49. The system of claim 35, wherein the first binding domain comprises a PD-1 binding domain of PD-L1 or PD-L2, or an antibody or an antibody derivative specific for PD-1.

50. The system of claim 49, wherein the first binding domain comprises a PD-1 agonist.

51. The system of claim 35, wherein the first binding domain comprises an A2AR binding agent.

52. The system of claim 51, wherein the A2AR binding agent comprises an antibody or a derivative thereof specific for A2AR.

53. The system of claim 51 or 52, wherein the A2AR binding agent comprises an A2AR agonist.

54. The system of any one of claims 35 to 53, wherein the non-CD19 pericyte surface protein and/or the non-CD19 vSMC surface protein comprise BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

55. The system of any one of claims 35 to 54, wherein the second domain comprises an antibody or a derivative thereof specific for BGN, FN1, SEMA5A, CD248, PDGFR-β, CD146, RGSS, NG2, αSMA, desmin, PLXDC1, THY1, CDH6, COL1A2, ITGA1, EDNRA, CSPG4, AXL, NTM, TNFRSF1A, S1PR3, or F3.

56. The system of any one of claims 35 to 55, wherein the agent is encoded in a vector.

57. The system of claim 56, wherein the vector is an expression vector having a promoter that is functional in a mammalian cell, and is operably linked to a nucleic acid that encodes the agent.

58. The system of any one of claims 35 to 57, wherein the agent is provided in a pharmaceutically acceptable formulation.

59. The system of claim 58, wherein the formulation is acceptable for intrathecal or intracerebral administration.

60. The system of any one of claims 35 to 55, wherein the agent is not cytotoxic to neurovascular pericytes and/or vSMCs.

61. A bispecific binding agent for reducing the potential neurotoxicity of a CD19-targeted therapy, wherein the bispecific binding agent comprises:
(a) a first binding domain that activates an immune checkpoint surface protein, and
(b) a second binding domain that specifically binds a non-CD19 neurovascular pericyte surface protein and/or a non-CD19 vSMC surface protein.

62. The bispecific binding agent of claim 61, wherein the immune checkpoint surface protein comprises PD-1, CTLA-4, ICOS, LAG-3, 2B4, BTLA, CD45, CD148, RPTPa, RPTPk, LAR, LYP/Pep, PTP-PEST, SHP-1, SHP-2, TCPTP, PTPH1, PTP-MEG1, PTP-BAS, PTP-MEG2, HePTP, MKP-1, PAC-1, MKP-2, MKP3, MPK-5, MKP-7, VHR, PTEN, LMPTP, or TIM-3.

63. The bispecific binding agent of claim 62, wherein the first binding domain comprises a CTLA-4-binding domain of CD80 or CD86.

64. The bispecific binding agent of claim 62, wherein the first binding domain comprises a CTLA-4 agonist.

65. The bispecific binding agent of claim 64, wherein the CTLA-4 agonist comprises an antibody or an antibody derivative.

66. The bispecific binding agent of claim 61 or 62, wherein the first binding domain comprises a PD-1 binding domain of PD-L1 or PD-L2, or an antibody or a derivative thereof specific for PD-1.

67. The bispecific binding agent of claim 66, wherein the first binding domain comprises a PD-1 agonist.

68. The bispecific binding agent of claim 61 or 62, wherein the first binding domain comprises an A2AR binding agent.

69. The bispecific binding agent of claim 68, wherein the A2AR binding agent comprises an antibody or a derivative thereof specific for A2AR.

70. The bispecific binding agent of claim 69, wherein the A2AR binding agent comprises an A2AR agonist.

71. The bispecific binding agent of any one of claims 61 to 70, wherein the non-CD19 pericyte surface protein and/or the non-CD19 vSMC surface protein comprises BGN, FN1, SEMA5A, CD248, PDGFR-β, CD146, RGS5, NG2, αSMA, desmin, PLXDC1, THY1, CDH6, COL1A2, ITGA1, EDNRA, CSPG4, AXL, NTM, TNFRSF1A, S1PR3, or F3.

72. A vector that encodes the bispecific binding agent of any one of claims 61 to 71.

73. The vector of claim 72, wherein the vector is an expression vector having a promoter that is functional in a mammalian cell, and is operably linked to a nucleic acid that encodes the bispecific binding agent.

74. A formulation for use in connection with CD19-targeted therapy, the formulation comprising:
(a) an effective amount of the bispecific binding agent of any one of claims 61 to 71 or the vector of any one of claims 72 to 73; and
(b) a pharmaceutically acceptable carrier.

75. The formulation of claim 74, wherein the effective amount is sufficient to reduce the number of neurovascular pericytes and/or vSMCs that are killed or incapacitated by the CD19-targeted therapy in vitro by about 10%, about 20%, about 30%, about 40%, about 50%, about 60%, about 70%, about 80%, about 90%, or about 100%.

76. The formulation of claim 74 or 75, wherein the agent reduces the number of neurovascular pericytes and/or vSMCs that are killed or incapacitated by the CD19-targeted therapy in an in vivo animal model by about 10%, about 20%, about 30%, about 40%, about 50%, about 60%, about 70%, about 80%, about 90%, or about 100%.

77. The formulation of any one of claims 74 to 76, wherein the agent reduces the disruption of the BBB by the CD19-targeted therapy by about 10%, about 20%, about 30%, about 40%, about 50%, about 60%, about 70%, about 80%, about 90%, or about 100%, as measured in an in vivo animal model using exclusion of a marker as a measure of BBB permeability.

78. The formulation of claim 77, wherein the BBB disruption is measured using Evans Blue dye exclusion.

79. A method for aiding in the treatment of a B-cell hyperproliferative disorder in a subject, the method comprising:
administering an effective amount of:
(a) the formulation of any one of claims 33 to 34 and 74 to 78;
(b) the system of any one of claims 35 to 60;
(c) the bispecific binding agent of any one of claims 61 to 70; or
(d) the vector of any one of claims 72 to 73.

80. A kit for the treatment of a B-cell hyperproliferative disorder in a subject, comprising:
(a) the formulation of any one of claims 33 to 34 and 74 to 78;
(b) the system of any one of claims 35 to 60;
(c) the bispecific binding agent of any one of claims 61 to 70; or
(d) the vector of any one of claims 72 to 73; and
(e) printed instructions for the use thereof.

81. The use of:
(a) the formulation of any one of claims 33 to 34 and 74 to 78;
(b) the system of any one of claims 35 to 60;
(c) the bispecific binding agent of any one of claims 61 to 70; or
(d) the vector of any one of claims 72 to 73;
for the treatment of a B-cell hyperproliferative disorder in a subject.

82. The use of:
(a) the formulation of any one of claims 33 to 34 and 74 to 78;
(b) the system of any one of claims 35 to 60;
(c) the bispecific binding agent of any one of claims 61 to 70; or
(d) the vector of any one of claims 72 to 73;
for the manufacture of a medicament for the treatment of a B-cell hyperproliferative disorder in a subject.
